(19) United States
(12) Patent Application Publication
(10) Pub. No.: US 2024/0060040 A1
(43) Pub. Date: Feb. 22, 2024

(54) METHODS FOR GENERATING INNER EAR HAIR CELLS

(71) Applicant: Ear Science Institute Australia, Subiaco (AU)

(72) Inventor: Yee Man Elaine Wong, Subiaco (AU)

(73) Assignee: Ear Science Institute Australia, Subiaco (AU)

(21) Appl. No.: 18/032,051

(22) PCT Filed: Oct. 14, 2021
(86) PCT No.: PCT/AU2021/051204

§ 371 Date: Apr. 14, 2023

(30) Foreign Application Priority Data
Oct. 14, 2020 (AU) 2020903734

(51) Int. Cl. C12N 5/0793; A61K 35/30; G01N 33/50

(52) U.S. Cl. CPC C12N 5/062; A61K 35/30; G01N 33/5058; C12N 2500/84; C12N 2501/15; C12N 2501/115; C12N 2501/155; C12N 2501/415; C12N 2501/41; C12N 2501/727; C12N 2506/45; C12N 2503/02

(57) ABSTRACT

The present invention relates to methods and compositions for producing differentiated otic cells. In particular, the invention relates to methods and compositions for the production of inner ear cells from pluripotent stem cells. The present invention also relates to methods of treating sensorineural hearing loss.

### FIELD OF THE INVENTION

This invention generally concerns methods and compositions for producing differentiated otic cells. In particular, the invention relates to methods and compositions for the production of inner ear cells from pluripotent stem cells.

### BACKGROUND

The following discussion of the background art is intended to facilitate an understanding of the present invention. The discussion is not an acknowledgement or admission that any of the material referred to is or was part of the common general knowledge as at the priority date of the application.

Sensorineural hearing loss (SNHL) is hearing loss whose cause lies in the inner ear or vestibulocochlear nerve. It accounts for approximately 90% of all reported hearing loss. Often, the cause of SNHL is damage to hair cells in the inner ear, which detect movement and sounds. Damage to hair cells in the inner ear can also cause loss of balance and/or dizziness. Many factors can cause damage to these hair cells, including genetic factors, environmental stimuli such as loud noises, as well as ototoxic drugs.

The inner ear, which is the innermost part of the vertebrate ear, comprises two main functional parts, being the cochlea and the vestibular system. The cochlea is dedicated to hearing. It is a spiral-shaped organ that converts the mechanical vibrations of the tympanic membrane and ossicles caused by sound into pressure waves in fluid, then into nerve impulses that are transmitted to the brain. The vestibular system is dedicated to balance. Both parts contain hair cells (inner ear hair cells).

The cochlea contains inner hair cells, which respond to sound by transforming the sound vibrations in the fluids of the cochlea into electrical signals to be carried by the auditory nerve to the brain, and outer hair cells, which mechanically amplify low-level sound that enters the cochlea for perception by the inner hair cells. Hair cells are located within the Organ of Corti of the inner ear cochlea and consist of one row of inner hair cells and three rows of outer hair cells. Sound detection is achieved by mechanostimulation of the stereociliary hair bundle structure located on the apical surface of each hair cell. Hair cells in mammals proliferate during development but lose capacity to regenerate shortly after birth, and therefore damage to these cells in children and adults is permanent and can cause irreparable hearing loss.

The vestibular system also contains hair cells (vestibular hair cells) that similarly transduce mechanical movement into electrical signals, which are interpreted in the brain as a sense of balance and spatial orientation.

Development of Inner Ear Hair Cells

The formation of correct hair cells and their hair bundle organelles requires the regulation of numerous developmental cues from signalling pathways. Many types of genetic deafness can be attributed to defects in signalling pathways during inner ear development and result in long term irreversible damage to hair cells.

The inner ear begins to develop in humans during about week 4 after conception. It is derived from a pair of sensory placodes, known as the otic placodes, which are thickenings on the ectoderm. The otic placodes fold inwards, forming a depression which then separates from the surface to form fluid-filled otic vesicles. The otic vesicle then differentiates into the various inner ear structures, including the cochlea and semi-circular canals. Otic vesicles in the early stage of development can be divided into the proneurogenic, and prosensory components. The neurogenic component gives rise to the auditory and vestibular neurons, the prosensory component (the otic prosensory vesicle) gives rise to the support cells and hair cells.

During inner ear development, the otic vesicle cells are committed to either sensory or non-sensory cell fates, which later contribute to the sensory hair cells and spiral ganglia as well as the non-sensory supporting cells. Regional specification of the otic vesicle is critical for directing the otic vesicle cells towards a prosensory fate. In the developing cochlea duct, the prosensory domain contains the progenitors of both sensory hair cells and non-sensory supporting cells which form the Organ of Corti. The Organ of Corti is a specialized sensory epithelium which runs the length of the cochlear duct, and is flanked by two nonsensory domains, the Greater Epithelial Ridge (GER) and the Lesser Epithelial Ridge (LER). Within the Organ of Corti, sensory hair cells are surrounded by non-sensory supporting cells, namely Hensen's cells, pillar cells and Deiters cells.

Various markers are involved in the specification of hair cells during development. Sox2, Eya1, Six1, Notch and FGF signalling are involved in the specification of cell fates in the Organ of Corti. The prosensory cells express Jagged2 (Jag2), Delta-like 1 (DII1), and Delta-like 3 (DII3), which lead to Notch pathway activation and inhibit hair cell fate through inhibition of the bHLH gene Atoh1, an inducer of hair cell differentiation. Inhibition of Notch signalling leads to an increase in Atoh1-positive hair cells.

The prosensory domain of the Organ of Corti is distinctively marked by the expression of cyclin-dependent kinase inhibitor P27kip1. The progenitor cells exit the cell cycle and terminate their proliferation from the apex towards the base of the cochlear duct between embryonic days 12 and 14 (mouse Embryonic stage of development E12.0 to E14.0). In the nascent Organ of Corti, hair cell differentiation initiates from the base towards the apex, as expression of the hair cell differentiation factor gene Atoh1 begins in the base of the cochlea between E13.5 and E14.5 and reaches the apex at around E17.5. Ectopic Atoh1 expression and hair cell regeneration from non-sensory GER regions in cochlea can be induced by over-expression of Eya1, Six1 and Sox2 in mouse explants.

The Sonic Hedgehog Pathway

The Sonic Hedgehog (SHH) signalling pathway is also involved in the development of otic cells. The SHH pathway regulates epithelial-mesenchymal interactions during the development of many organs. SHH protein is synthesised in epithelial cells and in many situations acts as a paracrine factor through its receptor PATCHED 1 (Ptch1) that is expressed in adjacent mesenchymal cells. Disruption of SHH-signalling has provided evidence for its important and diverse roles in organogenesis. SHH knockout mice exhibit various developmental defects, including cyclopia, neural tube defects and absence of distal limb structures. Inhibition of SHH-signalling using cyclopamine (CYC) has further demonstrated the role of SHH signalling in development of the neural tube, gastro-intestinal tract, pancreas, and in hair follicle morphogenesis.

The SHH-signalling pathway includes SHH, Cdo, Ptch1, Smoothened (SMO), GLI-1, GLI-2 and GLI-3. SHH is the ligand for a receptor complex which is made up of Cdo, Ptch1 and SMO. SMO is believed to transduce the signal and is a key element of the SHH signalling pathway. Gli-1, Gli-2 and Gli-3 are transcription factors.

Whilst there has been some research aimed at identifying the role of the SHH signalling pathway in the development and differentiation of inner ear hair cells, the precise role of each of the members of the hedgehog family is not known.

Inner Ear Organoids

There is a need to develop hair cells and their surrounding tissue in vitro, in order to study these structures, and for therapeutic uses.

Organoids are 3D cell aggregates that have the ability to form morphological and functional similarities to human organs. They can also be used for disease modelling, drug screening, tissue engineering, as well as the analysis of mutation mechanisms, due to their ability to regenerate and differentiate. The development of organoids resembling the cochlear hair cell and functional synapse can also be used to develop stem cell therapy treatments for hearing loss or deafness.

Pluripotent stem cells offer a possible approach to developing such models, as well as the production of inner ear hair cells for stem cell therapy.

Pluripotent stem cells are cells that can proliferate and differentiate into different cell types. Pluripotent stem cells include embryonic stem cells as well as induced pluripotent stem cells. Embryonic stem cells are derived from the undifferentiated inner mass cells of an embryo. Induced pluripotent stem cells are generated from adult cells by reprogramming somatic cells or differentiated progenitor cells to a state of pluripotency. Despite originating from somatic cells, induced pluripotent stem cells are capable of growing perpetually and differentiating into cells of the three germ layers.

Whilst differentiation of pluripotent stem cells can occur spontaneously, they can also be induced to differentiate through culturing the cells in the presence or absence of specific molecules that are involved in the differentiation process. The stage of differentiation and the identity of the cells throughout the differentiation process can be recognized by testing for the presence or absence of markers that are known to be present at the different stages of differentiation.

Recent studies have generated functional mechanosensitive vestibular or putative cochlear hair cells in organoids deriving from human induced pluripotent stem cells (hiPSC), as an alternative model to study genetic diseases and potential therapeutic approaches. For example, Koehler et al., Nature Biotechnology, (2017), 35(6) 583 reported the development of inner ear organoids and sensory epithelia innervated by sensory neurons from human embryonic stem cells and human induced pluripotent stem cells. In particular, Koehler et. al. 2017 reported the development of these tissues by using a three-dimensional culture system and modulating TGFβ (transforming growth factor-β), BMP (bone morphogenetic protein), FGF (Fibroblast growth factor) and WNT (Wingless Int-1) signalling to develop otic-vesicle-like structures. These vesicles developed over the course of 2 months into inner ear organoids with sensory epithelia. The organoid cells were reported in Koehler et. al. 2017 to have adopted a vestibular type II hair cell phenotype. Whilst the process described by Koehler et. al. 2017 produced inner ear hair cells, non-sensory or immature otic epithelia were preferentially induced. Koehler et. al. 2017 reported a seemingly low efficiency of hair cell induction.

U.S. Pat. No. 9,624,468, describes a method of generating inner ear tissues from pluripotent stem cells. In particular, it describes a method of generating mechanosensitive hair cells from human pluripotent stem cells comprising: (i) culturing pluripotent stem cells under conditions that result in formation of embryoid bodies from the cultured pluripotent stem cells; (ii) adding an extracellular matrix protein to the embryoid bodies; (iii) culturing the embryoid bodies in the presence of BMP2, BMP4, or BMP7 and a TGFβ inhibitor to form non-neural ectoderm; (iv) culturing the non-neural ectoderm formed in (iii) in the absence of the BMP4 and the TGFβ inhibitor, and in the presence of an exogenous FGF and a BMP inhibitor, in a floating culture, to generate preplacodal ectoderm; (v) culturing the preplacodal ectoderm, in a floating culture, in the absence of the exogenous FGF and BMP inhibitor to obtain otic placode and inner ear sensory hair cells differentiating from the otic placode; and (vi) culturing the preplacodal ectoderm in (v) in the presence of an activator of Wnt/β-catenin signalling. The inner ear sensory hair cells identified in this patent comprised Type II vestibular hair cells.

Cells at different stages of development are identified by reference to the markers they exhibit. The hiPSC in 9,624,468 showed characteristics of early otic cell markers including paired box 8 (PAX8) for otic placode; E-cadherin (ECAD) and N-cadherin (NCAD) for ectodermal characteristics; SOX2 for neuroectoderm and paired box 2 (PAX2) for otic vesicles. Expression of these markers were reported by Koehler to demonstrate the early stages of inner ear development (Koehler et al., 2017).

Mature putative cochlear hair cells were also found in mature organoids after Day 35, resembling the stage of cochlear hair cell differentiation of the inner ear development (Jeong et al., Cell Death and Disease (2018) 9:922).

However, the generation of inner ear hair cells, and in particular inner hair cells, remains difficult and there is scope for increasing the efficiency of hair cell differentiation in the organoid models that have been described to date. It is an object of the present invention to overcome the shortcomings of the prior art.

### SUMMARY OF THE INVENTION

The present invention is based on an unexpected finding that inhibiting the Sonic Hedgehog pathway during the development of inner ear cells can improve the efficiency of inner ear hair cell differentiation. In particular the invention provides, inter alia, methods for the generation of inner ear hair cells, and in particular inner hair cells, using organoid models that have been described to date.

In a first aspect, the invention provides a method for producing inner ear hair cells comprising the steps of:
A. culturing otic prosensory vesicles in the presence of a SHH inhibitor in an amount sufficient to partially inhibit the SHH pathway and a culture medium comprising 5-10% of the gelatinous protein mixture secreted by EHS mouse sarcoma cells;
B. removing the SHH inhibitor from the culture in step A; and
C. culturing the cells in step B in a culture medium comprising 5-10% of the gelatinous protein mixture secreted by EHS mouse sarcoma cells to form inner ear hair cells.

Preferably steps A to C in the first aspect of the invention occur over a specific time period. Most preferably, step A occurs for about 10 days for otic placode and otic vesicles formation; step B occurs for about 15 days for sensory epithelium formation; and step C occurs for about 68 days for hair cell and neural innervation formation until maturation.

Preferably, the invention provides a method for producing inner ear hair cells, comprising the steps of:
AA. culturing pluripotent stem cells for about 21 days under conditions that result in the formation of otic prosensory vesicles;
A. culturing the otic prosensory vesicles produced in step AA in the presence of a sufficient amount of SHH inhibitor to partially inhibit the activity of the SHH pathway for about 15 days, in a culture medium containing 5-10% of the gelatinous protein mixture secreted by Engelbreth-Holm-Swarm (EHS) mouse sarcoma cells;
B. removing the SHH inhibitor from the culture in A; and
C. culturing the cells in B for at least 1 day in a culture medium comprising 5-10% gelatinous protein mixture secreted by Engelbreth-Holm-Swarm (EHS) mouse sarcoma cells to form inner ear hair cells.

In a preferred form of the first aspect, the invention provides a method for producing inner ear hair cells, comprising the steps of:
AA1. culturing induced pluripotent stem cells under conditions that result in the formation of embryoid bodies from the cultured pluripotent stem cells;
AA2. culturing the embryoid bodies from step AA1 in the presence of a FGF at a concentration of 2 to 4 ng/mL, and a TGF-β inhibitor at a concentration of 5 to 10 μM to form non-neural ectoderm cells;
AA3. culturing the non-neural ectoderm cells from step AA2 in the presence of FGF at a concentration of 50-100 ng/mL and a BMP inhibitor at a concentration of 100 to 200 nM to form pre-otic placodal epithelial cells;
AA4. culturing the pre-otic placodal epithelial cells from step AA3 in the presence of a WNT agonist at a concentration of 2 to 3 μM and a cell culture medium comprising 5 to 10% of the gelatinous protein mixture secreted by Engelbreth-Holm-Swarm (EHS) mouse sarcoma cells to form otic prosensory vesicles;
A. culturing the otic prosensory vesicles from step AA4 in the presence of a sufficient amount of SHH inhibitor to partially inhibit the activity of the SHH pathway, in a culture medium comprising 5-10% of the gelatinous protein mixture secreted by Engelbreth-Holm-Swarm (EHS) mouse sarcoma cells;
B. removing the SHH inhibitor from the culture in step A; and
C. culturing the cells from step B in a culture medium comprising 5-10% of the gelatinous protein mixture secreted by Engelbreth-Holm-Swarm (EHS) mouse sarcoma cells to form inner ear hair cells.

Steps AA1 to AA4 in the preferred form of the first aspect of the invention result in the production of otic prosensory vesicles from pluripotent stem cells. In step AA1, the pluripotent stem cell is cultured in conditions that result in the formation of embryoid bodies. In an embodiment, the pluripotent stem cells are cultured together with a ROCK inhibitor, Y-27632, to induce the production of embryoid bodies.

In step AA2 the embryoid bodies from step AA1 are cultured in the presence of a low concentration of FGF and TGF-β inhibitor in order to produce non-neural ectoderm cells. From this step onwards, the cell aggregates are known as “organoids”. Preferably, the FGF used in step AA2 is selected from any of FGF2, FGF3 or FGF10. In a particularly preferred embodiment, the FGF is FGF2. Preferably, the FGF is present at a concentration of 2-4 ng/mL. Preferably, the TGF-β inhibitor is SB-431542. Preferably, the TGF-β inhibitor is present at a concentration of 5-10 μM. In some embodiments of the first aspect of the invention, step AA2 additionally comprises culturing the embryoid bodies in the presence of BMP.

In step AA3 the non-neural ectoderm cells are cultured in the presence of a BMP inhibitor and a high concentration of FGF to form pre-placodal otic epithelial cells. Preferably, the BMP inhibitor is LDN-193189. Preferably, the FGF used in steps AA3 is selected from any of FGF2, FGF3 or FGF10. In a particularly preferred embodiment, the FGF is FGF2. Preferably, the FGF is present at a concentration greater than that in step AA2. In a particularly preferred embodiment, the FGF is present at a concentration of 50-100 ng/mL.

In step AA4 in the preferred form of the first aspect of the invention the pre-placodal otic epithelial cells are cultured in the presence of a WNT agonist in a culture medium comprising 5-10% of the gelatinous protein mixture secreted by Engelbreth-Holm-Swarm (EHS) mouse sarcoma cells in order to produce prosensory otic prosensory vesicles. Preferably, the WNT agonist is CHIR-99021.

The inventor has identified that the inhibition of the SHH pathway through the treatment of otic prosensory vesicles with a SHH inhibitor can increase the efficiency of differentiation of inner ear hair cells. Steps A to C in the preferred form of the first aspect of the invention relate to culturing the otic prosensory vesicles in the presence of a SHH inhibitor.

The SHH inhibitor used in the method of the invention can inhibit any molecule in the SHH pathway. Preferably, the SHH inhibitor is selected from cyclopamine (CYC) (CAT 239803 from Merck Millipore), GANT58 (CAT 73984 from STEM CELL Technologies) or GANT61 (CAT 73692 from STEM CELL Technologies). The SHH inhibitor is present in an amount sufficient to partially inhibit the activity of the SHH pathway. Preferably, the SHH inhibitor inhibits the activity of the SHH pathway from 50% to 70%. In a particularly preferred embodiment, the SHH inhibitor is cyclopamine and it is present in step A in the preferred form of the first aspect of the invention at a final concentration of 1-2 μM. In another preferred embodiment, the SHH inhibitor is GANT61 and is present in step A at a final concentration of 1-2 μM. In another preferred embodiment, the SHH inhibitor is GANT58 and is present in step A at a final concentration of 1-2 μM.

In steps B and C in the preferred form of the first aspect of the invention, the SHH is removed from the cell culture medium, but the cells continue to be cultured in medium containing 5-10% of the gelatinous protein mixture secreted by EHS mouse sarcoma cells until maturation. Preferably, the cells are cultured using the hanging drop method. Most preferably, the cells are cultured without shaking.

Preferably, the steps AA2 to C in the preferred form of the first aspect of the invention occur over a specific timeline. Preferably, the timeline mimics the time taken to reach each stage in vivo. Most preferably the steps AA2 to C occur in accordance with the following timeline:
(i) step AA2 occurs from day 0 to day 3, where day 0 is the day on which step AA2 commences for non-neural ectodermal formation;
(ii) step AA3 occurs from day 4 to day 7 for early pre-otic placodal epithelium formation;
(iii) step AA4 occurs from day 8 to day 17 for otic placode and otic vesicles formation;
(iv) step A occurs from day 18 to day 32 for sensory epithelium formation; and
(v) step C occurs from day 33 onwards for hair cell and neural innervation formation until maturation.

Preferably, maturation occurs between day 60-200.

The pluripotent stem cell can be from any organism. Preferably, the pluripotent stem cell is a human pluripotent stem cell. In a further preferred embodiment, the pluripotent stem cell is an induced human pluripotent stem cell. The induced pluripotent stem cells can be from any cell line.

The inner ear hair cells produced by the methods of the invention can be of any type. In a particularly preferred embodiment, the inner ear hair cell is an inner hair cell.

In a second aspect, the invention comprises a composition comprising inner ear hair cells produced by the methods of the invention. In some embodiments, the composition can additionally comprise other agents, such as preserving agents. Preferably, the composition additionally comprises one or more pharmaceutically acceptable agents.

In a third aspect, the invention comprises a method of treating a subject suffering from sensorineural hearing loss by administering a composition comprising inner ear hair cells produced by the methods of the invention.

In a fourth aspect, the invention comprises a method for assessing the ototoxicity or therapeutic effectiveness of a test agent comprising the step of treating a population of inner ear hair cells or an organoid comprising inner ear hair cells produced by the methods of this invention, with a test agent and measuring the effect of the test agent on the cells or organoid.

In a fifth aspect, the invention comprises a method of diagnosing an otological disease in a patient comprising the step of evaluating for the presence of absence of a marker specific for the disease in a population of patient derived inner ear hair cells or a patient derived organoid comprising inner ear hair cells produced by the methods of this invention.

In a sixth aspect, the invention comprises the use a composition comprising inner ear hair cells produced by the methods of the invention in the manufacture of a medicament for the treatment of sensorineural hearing loss in a subject in need thereof.

In a seventh aspect, the invention comprises a method of regenerating inner ear hair cells in a subject comprising the step of administering a SHH inhibitor to the subject's inner ear. Preferably, the SHH inhibitor is selected from cyclopamine (CYC) (CAT 239803 from Merck Millipore), GANT58 (CAT 73984 from STEM CELL Technologies) or GANT61 (CAT 73692 from STEM CELL Technologies). The SHH inhibitor is present in an amount sufficient to partially inhibit the activity of the SHH pathway. Preferably, the SHH inhibitor inhibits the activity of the SHH pathway from 50% to 70%. In a particularly preferred embodiment, the SHH inhibitor is administered at a concentration of 1-2 μM.

In an eighth aspect, the invention comprises inner ear hair cells produced by the methods of the invention.

In a ninth aspect, the invention comprises an organoid comprising inner ear hair cells produced by the methods of the invention.

In a tenth aspect, there is provided a method of enhancing inner ear hair cell differentiation in a subject comprising the step of partially inhibiting Cdo expression in the subject. Preferably, Cdo expression is inhibited by the administration of siRNA or CRISPR in a therapeutically effective amount to the subject.

Other aspects and advantages of the invention will become apparent to those skilled in the art from a review of the ensuing description, which proceeds with reference to the following illustrative drawings.

### BRIEF DESCRIPTION OF THE FIGURES

The figures provided below illustrate the invention in the following preferred embodiments and the Examples.

FIG. 1 provides an overview of the steps in a preferred method of the invention. In particular it presents a protocol of the invention using an exemplar agent to promote hair cell differentiation of human iPS cells.

FIG. 2 illustrates extra formation of hair cells and expansion of supporting cells by using hair cell marker MyosinVIIa and supporting cell marker Sox2 in the Organ of Corti of E16.5 Cdo^{-/-} mutants in 10× magnification.

FIG. 3 illustrates supernumerary hair cells in Shh^{+/-}; Cdo^{-/-} compound mutant cochlea using hair cell marker MyosinVIIa and neural marker beta-tubulin III Tuj1 antibodies to mark the hair cells and nerve innervation into cochlea at E16.5 in 10× magnification.

FIG. 4 illustrates ectopic hair cells with nerve innervation in Shh^{+/-}; Cdo^{-/-} compound mutant cochlea using hair cell marker MyosinVIIa and neural marker Tuj1 antibodies to mark the hair cells and nerve innervation into E16.5 cochlea in 20× magnification.

FIG. 5 illustrates the absence of Pillar cells in Cdo^{-/-} mutants using pillar cell specific marker P75NTR to mark the pillar cells in cochlea at E16.5 in 10× magnification.

FIG. 6 illustrates Cdo expression in supporting cells in mouse cochlea at E16.5.

FIG. 7 illustrates prosensory domain specification by Sox2 in Cdo and Cdo/Shh compound mutant cochlea at E14.5 from basal to apical regions.

FIG. 8 illustrates cell cycle exit by P27Kip1 in Cdo and Cdo/Shh compound mutant cochlea at E14.5 from basal to apical regions.

FIG. 9 illustrates Gli gene expression in SHH pathway in mouse cochlea at E13.5 and E16.5.

FIG. 10 illustrates gross morphology of Human iPSCs derived inner ear organoid at Day 1-10 in 4× and 10× magnification.

FIG. 11 illustrates gross morphology of Human iPSCs derived inner ear organoids at Day 20-40 in 10× magnification.

FIG. 12 illustrates ectodermal cell fate by using ECAD and PAX2 in the human iPSCs derived inner ear organoids at Day 20 in 10× magnification.

FIG. 13 illustrates otic identity by using NCAD and SOX2 in the human iPSCs derived inner ear organoid at Day 40 in 10× magnification.

FIG. 14 illustrates hair cells with nerve innervation in human iPSCs derived inner ear organoid at Day 60 using hair cell marker MyosinVIIa and neural marker Tuj1 antibodies to mark the hair cells and nerve innervation in 10× magnification.

FIG. 15 illustrates cell fate analysis on human iPSCs derived inner ear organoid at Day 60 by single cell RNA sequencing.

FIG. 16 illustrates TaqMan® gene expression assays on human iPSCs derived inner ear organoid at Day 20 and Day 60 by quantitative real-time polymerase chain reaction (qRT-PCR) analysis.

FIG. 17 illustrates MyosinVIIa and Tuj1 expression in histological section of organoids at Day 60 treated with cyclopamine, GANT58 and GANT61 as observed using confocal microscopy in 20× magnification.

FIG. 18 illustrates SOX2 and Tuj1 expression in histological section of organoids at Day 60 treated with cyclopamine, GANT58 and GANT61 as observed by using confocal microscopy in 20× magnification.

### DETAILED DESCRIPTION OF THE INVENTION

The present invention is directed to improved methods and compositions for generating inner ear hair cells from pluripotent stem cells. The invention is based on the unexpected discovery that inhibiting the Sonic Hedgehog pathway during the development of inner ear cells can improve the efficiency of inner ear hair cell differentiation.

For convenience, the following sections generally outline the various meanings of the terms used herein. Following this discussion, general aspects of the invention are discussed, followed by specific examples demonstrating the properties of various embodiments of the invention.

### Definitions

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however, be understood to be common general knowledge.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### General Embodiments

According to the invention the inventor has revealed that inhibiting the SHH pathway by adding a SHH inhibitor to the cell culture at a specific stage during the production of inner ear hair cells from otic prosensory vesicles or pluripotent stem cells can increase the efficiency of hair cell differentiation.

The hair cells derived from the methods of this invention exhibit functional properties of native mechanosensitive hair cells and, in some embodiments, also present in situ innervation of hair cells.

Steps AA1 to AA4—Production of Otic Prosensory Vesicles

Steps AA1 to AA4 of the present method describe the production of otic prosensory vesicles from pluripotent stem cells, which ultimately can give rise to support cells (non-sensory) and hair cells (sensory).

The pluripotent stem cells used in the invention can be embryonic stem cells or induced pluripotent stem cells. The cells may be from any organism. Preferably, the pluripotent stem cells are human pluripotent stem cells. More preferably, the pluripotent stem cells are human induced pluripotent stem cells. The human induced pluripotent stem cells can be patient specific.

The pluripotent stem cells can be of any cell line. Preferably, the pluripotent stem cell is of the fibroblast cell line Gibco Human Episomal iPSC line, Thermo Fisher A18945.

Step AA1

Step AA1 comprises culturing pluripotent stem cells under conditions that result in the formation of embryoid bodies from the cultured pluripotent stem cells. Most preferably, the pluripotent stem cells are of human origin.

Preferably, the pluripotent stem cells are induced pluripotent stem cells (iPSCs) and are cultured in a suitable medium, such as mTeSR™1® medium (CAT 85850 STEM CELL Technologies) as was used in Koehler et. al. 2017, together with a ROCK inhibitor (Y-27632) for about 3 to 4 days to form three dimensional embryoid bodies. Preferably, the amount of ROCK inhibitor used is 10-20 μM. The cells can be incubated for up to 2 days before commencing step AA2.

Step AA2

Otic tissues arise from the non-neural ectoderm during development. Step AA2 comprises forming non-neural ectoderm from the embryoid bodies produced in step AA1. Preferably, step AA2 commences on day 0, and occurs until day 3, that is, step AA2 occurs over about 4 days. Most preferably, the cells are cultured in a 6-well suspension culture plate during step AA2.

Preferably, the embryoid bodies are transferred to a chemically defined differentiation medium containing FGF and TGFβ inhibitor. The presence of FGF and the TGFβ inhibitor together stimulates the epithelization and ectoderm differentiation on the embryoid body surface.

The FGF (fibroblast growth factor) family is a group of structurally related polypeptide growth factors. In the mammalian system, there are 22 members of the FGF family. Preferably, the FGF is FGF2, FGF3 or FGF10. Most preferably, the FGF is FGF2, which is known to have a specific function in inner ear development. The presence of FGF2 is sufficient to induce early pre-otic placode epithelium formation.

When FGF2 is present, it is preferably present in a low concentration to act as an inducer for non-neural ectoderm formation. Preferably, the concentration of FGF2 is below about 4 ng/mL. The concentration of FGF2 may be selected from the list of 0.5 ng/mL, 1 ng/mL, 2 ng/mL, 3 ng/mL and 4 ng/mL. Most preferably, the final concentration of the FGF2 in the medium is about 2-4 ng/mL.

The presence of the TGFβ inhibitor has been shown to induce non-neural markers, and therefore induce non-neural ectoderm formation. In some embodiments, the TGFβ inhibitor is selected from SB 431542 (CAS No. 301836-41-9), A 83-01 (CAS No. 909910-43-6), GW 788388 (CAS No. 452342-67-5), LY 364947 (CAS No. 396129-53-6), RepSox (CAS No. 446859-33-2), SB 505124 (CAS No. 694433-59-5), SB 525334 (CAS No. 356559-20-1), or SD 208 (CAS No. 356559-20-1) at a concentration of 0.1 μM to 100 μM. Preferably, the TGFβ inhibitor is SB-431542 and is present in a concentration of 2 μM to 20 μM, 2.5 μM to 12.5 μM, 1 μM to 15 μM, or, most preferably about 5-10 μM.

In some preferred embodiments of the invention, the chemically defined differentiation medium in step AA2 additionally comprises gelatinous protein mixture secreted by Engelbreth-Holm-Swarm (EHS) mouse sarcoma cells and an FGF. This is to provide structure for non-neural ectoderm and pre-otic placode epithelium formation.

Preferably, the gelatinous protein mixture secreted by Engelbreth-Holm-Swarm (EHS) mouse sarcoma cells is Matrigel® (CAT A356231, Corning). In a preferred embodiment, the concentration of gelatinous protein mixture secreted by Engelbreth-Holm-Swarm (EHS) mouse sarcoma cells is low, being about 5-10%. In a particularly preferred embodiment, the gelatinous protein mixture secreted by Engelbreth-Holm-Swarm (EHS) mouse sarcoma cells is present in a concentration of 10%.

In other embodiments, the chemically defined differentiation medium in step AA2 additionally comprises a BMP (bone morphogenetic factor). The differentiation of the embryoid bodies into non-neural ectoderm can require the presence of BMP activity. In some cell lines, such as Gibco iPSC cell lines, endogenous BMP activity can be sufficient for non-neural specification, and no further BMP needs to be added to the medium to induce non-neural ectoderm formation. However, in other cell lines, it may be necessary to add a BMP to induce non-neural ectoderm formation. Preferably, the BMP is selected from BMP2, BMP4, or BMP7. Preferably the BMP is BMP4. The concentration of BMP used in the method can range from at least about 1 ng/ml to about 50 ng/mL, e.g., about 2 ng/mL, 4 ng/mL, 5 ng/mL, 7 ng/mL, 12 ng/mL, 15 ng/mL, 20 ng/mL, 25 ng/mL, 32 ng/mL, 40 ng/mL, or another concentration of a BMP from at least about 1 ng/mL to about 50 ng/mL. In some embodiments, the BMP to be used is BMP4 at a concentration of about 2.5 ng/mL BMP4.

The formation of the non-neural ectoderm is characterised by the presence of non-neural ectoderm markers such as TFAP2A and DLX3 and the absence of neuroectodermal markers such as PAX6 and N-cadherin. In some embodiments, the formation of the non-neural ectoderm is confirmed by screening for the presence of one or more non-neural ectoderm markers before commencing step AA3.

Step AA3

Step AA3 comprises forming pre-otic placodal epithelium from the non-neural ectoderm cells in step AA2. Preferably, step AA3 is conducted over days 4-7, that is, step AA3 occurs over about 4 days.

During step AA3, the non-neural ectodermal cells are treated with FGF and a BMP inhibitor. FGF activation and BMP inhibition is necessary for pre-placode and otic induction from non-neural ectoderm cells.

Preferably, the FGF is FGF2, FGF3 or FGF10. The FGF is present in a higher final concentration than in step AA2. Preferably, the FGF is present in a concentration of 5 ng/mL to 100 ng/mL. Most preferably, the FGF is FGF2 and is present at a final concentration of about 50-100 ng/mL. The concentration of FGF used in the method can range from at least about 40 ng/mL to about 100 ng/mL, e.g., about 40 mg/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, or another concentration of a FGF from at least about 50 ng/mL to about 100 ng/mL. Most preferably, the FGF is present at a final concentration of about 50 ng/mL. Most preferably, the FGF is FGF2 and is present at a final concentration of 50 ng/mL.

The BMP inhibitor is selected from the list of LDN-193189 (CAS No. 1062368-24-4), DMH1 (CAS No. 1206711-16-1) or Dorsomorphin (CAS No. LDN-193189). Preferably, the BMP inhibitor is LDN-193189. Preferably, the BMP inhibitor is present at a concentration of 100-200 nM. The concentration of BMP used in the method can range from at least about 100 nM to about 200 nM, e.g., 100 nM, 110 nM, 120 nM, 130 nM, 140 nM, 150 nM, 160 nM, 170 nM, 180 nM, 190 nM, 200 nM, or another concentration of BMP from 100 nM to 200 nM. Most preferably, the BMP inhibitor is present at a concentration of about 200 nM.

The formation of pre-otic placodal epithelium is characterised by the presence of markers such as PAX8, SOX2, TFAP2A, ECAD and NCAD. In some embodiments, the formation of the pre-otic placodal epithelium is confirmed by screening for the presence of one or more non-neural ectoderm markers before commencing step AA4.

Step AA4

Step AA4 comprises otic prosensory vesicle formation from the pre-otic placodal epithelium in step AA3. Preferably, step AA4 is conducted over days 8-17, that is, step AA4 occurs over about 10 days.

Pre-otic placodal epithelium can develop into otic tissue or alternatively, epibranchial tissue. The activation of the WNT pathway has been shown to be important for otic, but not epibranchial development. Accordingly, during step AA4, the pre-otic placodal epithelium from step AA3 is treated with a WNT agonist.

In some embodiments, the WNT agonist is a Gsk3 inhibitor. In some embodiments, the Gsk3 inhibitor is selected from the group consisting of CHIR 99021, CHIR 98014, BIO-acetoxime, LiCl, SB 216763, SB 415286, AR A014418, 1-Azakenpaullone, and Bis-7-indolylmaleimide. Preferably, the WNT agonist is CHIR 99021. In a preferred embodiment, the WNT agonist is present at a concentration of at least about 1 μM to about 10 μM in the medium, e.g., 1.0 μM, 1.5 μM, 2.0 μM, 2.5 μM, 3 μM, 4 μM, 5 μM, 7 μM, 8.5 μM, 1.5 μM to 5 μM, 2 μM to 4 μM, or another concentration from about 2 μM to about 10 μM. Most preferably, the WNT agonist is CHIR 99021 and is present in a final concentration of 2-3 μM.

Preferably, from days 8-11, the cells are treated with the WNT agonist in a 6-well suspension culture plate for otic placode formation, and then on day 12, the resulting organoids are resuspended in an organoid maturation medium containing a gelatinous protein mixture secreted by Engelbreth-Holm-Swarm (EHS) mouse sarcoma cells (e.g. Matrigel®). Preferably, the medium is replaced after incubating the organoids for one hour, to allow the gelatinous protein mixture secreted by EHS mouse sarcoma cells (e.g. Matrigel®) to set. Preferably, the Matrigel® is present at a concentration of 0.1% to 20%, 1% to 15%, or 2.5% to 12.5%. Most preferably, the Matrigel is present at a concentration of 5-10%.

Preferably, the WNT agonist is then added to the culture on days 12-14, preferably at a concentration of about 2-3 μM, in order to induce the formation of otic pits. The formation of otic pits can be characterised by the presence of markers such as PAX2, PAX8, SOX2, SOX10 and JAG1. The organoids are cultured until day 17 in the presence of the WNT agonist to form otic prosensory vesicles. In some embodiments, the formation of the otic prosensory vesicles is confirmed by screening for the presence of one or more otic pit markers before commencing the next step.

Steps A to C—Inhibition of SHH Pathway

In an aspect of the invention, there is provided a method of producing inner ear hair cells comprising the treatment of otic prosensory vesicles with a SHH inhibitor and culturing the treated cells until maturation in order to form inner ear hair cells.

The present inventor has found that inhibiting the SHH pathway at a specific point during the development of inner ear hair cells increases the efficiency of inner ear hair cell differentiation. In particular, the inhibition of SHH signalling during the hair cell differentiation phase (after the early hair cell proliferation and growth phase) has been found by the present the inventor to increase the efficiency of inner ear hair cell differentiation.

Without being bound by theory, Hedgehog signaling in prosensory cells is thought to be activated by the Hedgehog ligand SHH, which is transiently produced by spiral ganglion neurons during cochlear outgrowth.

It is believed that at early stage inner ear development, the auditory cell fates in otic vesicles are established by the direct action of SHH. This is the stage where the proginetor cells are growing. The inner ear morphogenesis and auditory compartment, including Kölliker's organ and spiral ganglion cells are formed with the activation of Hedgehog signaling. The activation of Hedgehog signaling plays different roles in inner ear morphogenesis, cochlear progenitor cell proliferation and prosensory formation. Studies have shown that adding SHH signalling pathway agonists at this early stage promotes otic cells formation, proliferation and growth.

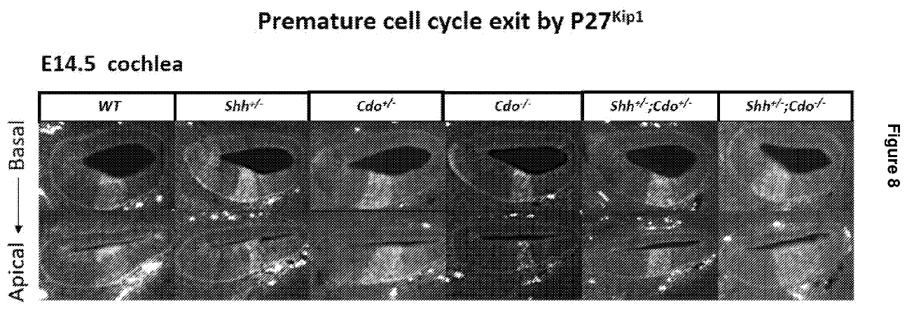

However, the role of the SHH pathway in later stages of inner ear development, after the prosensory stage, including the hair cell differentiation stage is not established. The differentiation stage is the stage where the cells adopt supporting, hair cell or neuronal configurations. The present inventor has identified that the inhibition of HH signalling promotes differentiation of hair cells and induces prosensory cells to drop out of the cell cycle prematurely, and to instead differentiate into hair cells. Without being bound by theory, the differentiation from progenitor cells into hair cells upon SHH pathway inhibition may involve the upregulation of Atoh1, leading to hair cell differentiation as shown in FIGS. 7-8 and 12-18.

The SHH pathway is critically involved in regulating development of inner ear hair cells. In particular, the SHH pathway may be involved in inducing the differentiation of otic vesicles into non-sensory cells or maintaining their non-sensory fates, and in restricting the prosensory domain within the Organ of Corti. Mutants where molecules involved in the SHH pathway have been knocked out display additional hair cell differentiation and reduced supporting cell differentiation. Accordingly, without being bound by theory, the inhibition of the SHH pathway with an inhibitor after otic vesicle formation reduces the differentiation of the otic vesicles to a non-sensory fate (i.e. supporting cells), and increases the efficiency of differentiation into a sensory fate, namely differentiation to inner ear hair cells.

However, complete inhibition of the SHH pathway is not desirable, as this can result in the lack of the development of many inner ear structures. The SHH pathway must therefore be partially inhibited. Preferably, the SHH pathway is inhibited by between about 50% to 70%.

Cdo (Cell adhesion molecule-related, down-regulated by oncogenes) is a novel receptor of the Hedgehog SHH pathway. Mutations in Cdo cause holoprosencephaly, a human congenital anomaly defined by forebrain midline defects prominently associated with diminished SHH pathway activity. Cdo functions as a component and target of the SHH signalling and feedback network. Cdo enhances SHH signalling by acting as a co-receptor with Ptch1, or via regulation of Gli transcription factors. A proper balance of Gli repressor and activators is required to mediate SHH signalling during inner ear morphogenesis. Cdo homozygous knockout mice have profound hearing loss. The inventor has surprisingly found that Cdo homozygous and SHH heterozygous knockout mice demonstrate increased inner hair cell differentiation compared with wild-type mice, suggesting a possible mechanism of action for how SHH inhibitors may act to increase inner ear hair cell differentiation in the present invention. The inventor has also identified that Cdo homozygous and SHH heterozygous knockout mice exhibiting increased hair cell differentiation show inhibition of SHH pathway between 50% to 70%.

The term ‘SHH inhibitor’ refers to an agent that can inhibit any molecule in the SHH pathway. For example, the SHH inhibitor may inhibit Smoothened (SMO), GLI transcription factors or SHH itself. Preferably, the SHH inhibitor is selected from the list of: cyclopamine (SMO inhibitor), GANT61 (GLI inhibitor), GANT58 (GLI inhibitor), CDO (SHH inhibitor), Vismodegiband (SMO inhibitor), Erismodegib (SMO inhibitor), arsenic trioxide (GLI inhibitor), IPI-929 (SMO inhibitor), BMS-833923/XL139 (SMO inhibitor), PF-04449913 (SMO inhibitor), LY2940680 (SMO inhibitor), RU-SKI (SHH inhibitor), or the anti-SHH monoclonal antibody 5E1 (SHH inhibitor). Most preferably, the SHH inhibitor is cyclopamine (SMO inhibitor), GANT58 or GANT61 (GLI inhibitor).

In a preferred embodiment, the SHH inhibitor is added to a culture medium containing otic prosensory vesicles. Preferably, the otic prosensory vesicles are produced using steps AA1 to AA4 described above. The inventor has found that the timing of the addition of the SHH inhibitor can be particularly important in enhancing inner ear hair cell differentiation. Preferably, the SHH inhibitor is added to a culture medium containing otic prosensory vesicles (preferably from step AA4) after the prosensory stage. Most preferably, the SHH inhibitor is added on day 18 from the commencement of step AA2. Addition of the SHH inhibitor at around day 18 is particularly important where the cells are of human origin.

The SHH inhibitor is added in an amount to partially inhibit the activity of the SHH pathway. Preferably, the SHH inhibitor is present in a concentration of 0.5 to 20 μM e.g., 0.5, 1 μM, 1.5 μM, 2 μM, 2.5 μM, 3 μM, 4 μM, 5 μM, 7 μM, 8 μM, 9 μM, 10 μM, 11 μM, 12 μM, 13 μM, 14 μM, 15 μM, 16 μM, 17 μM, 18 μM, 19 μM, 20 μM or another concentration from about 0.5 μM to about 20 μM.

In a preferred embodiment, the SHH inhibitor is cyclopamine and is present in a final concentration of about 0.5 μM to 3 μM. More preferably, the SHH inhibitor is cyclopamine and is present in a final concentration of 0.5 μM-2 μM. Most preferably, the cyclopamine is present in a final concentration of 1 μM. In another preferred embodiment, the SHH inhibitor is GANT61 and is present in a final concentration of about 0.5 μM to 20 μM. More preferably, the SHH inhibitor is GANT61 and is present in a final concentration of 1 μM-3 μM. Most preferably, the GANT61 is present in a final concentration of 2 μM. In another preferred embodiment, the SHH inhibitor is GANT58 and is present in a final concentration of about 0.5 μM to 3 μM. More preferably, the SHH inhibitor is GANT58 and is present in a final concentration of 0.5 μM-2 μM. Most preferably, the GANT58 is present in a final concentration of 1 μM. In another embodiment, the SHH inhibitor inhibits Cdo in the SHH pathway. Preferably, the inhibitor of Cdo inhibits the expression of Cdo and is a siRNA or CRISPR.

In step A, the SHH inhibitor is added together with a gelatinous protein mixture secreted by EHS mouse sarcoma cells (e.g. Matrigel®). Preferably, the Matrigel® is present at a concentration of about 5-10%. The presence of Matrigel® at a concentration of about 5-10% allows for the improved suspension of the organoids, for example those from step AA4. The improved suspension of the organoids allows for the cells to be cultured using the hanging drop method known in the art without shaking to sit the organoid in the medium. This has the advantage of not drying out as quickly and offering a better view of cell motility. The particular concentration of Matrigel used (5-10%) has the advantage of being sufficiently strong to hold the three-dimensional organoid. Preferably, the Matrigel is present at a concentration of 0.1% to 20%, 1% to 15%, or 2.5% to 12.5%. Most preferably, the Matrigel is present at a concentration of 5-10%.

Preferably, the cells are cultured in step A for about 10 days. More preferably, the cells are cultured until day 32 (from the commencement of step AA2) for sensory epithelium formation.

In step B, the SHH Inhibitor is removed from the medium. Preferably, the SHH is removed by washing. Most preferably, the cells remain in cell culture medium containing about 5-10% Matrigel®.

In a preferred embodiment, step C occurs on day 33 (from the commencement of step AA2) for at least 1 day.

In step C, the cells are preferably cultured in a cell culture medium containing about 5-10% Matrigel® until maturation. Preferably the cell culture medium also comprises Organoid Maturation Medium which is a serum-free cell culture medium for efficient establishment for long-term maintenance of organoid culture.

Preferably, the organoids are cultured until maturation. In an embodiment, maturation occurs at between 60-200 days from the commencement of step AA2. Preferably, maturation occurs by day 60-100. Maturation is identified by assessing the morphology of the cells.

Preferably, the organoids are left to mature in individual wells of 48-well suspension plates containing 5-10% Matrigel® with 1 mL Organoid Maturation Medium. Preferably, 200 μL of medium is changed daily for each well. Most preferably, the organoids are cultured using the hanging drop method and are not shaken during culture.

In some embodiments, the inner ear hair cells are detected after day 35 from the commencement of step AA2. Preferably, the inner ear hair cells are inner hair cells. Most preferably, the inner ear hair cells also exhibit in situ innervation. The presence of inner ear hair cells and neural innervation can be identified by immunostaining using MyoVIIa hair cell and TuJ1 nerve cell markers. The population of otic cell fate in organoid can be identified by single-cell RNA sequencing analysis.

### Selection Criteria

The organoids developed by the process described herein can be imaged and analysed throughout the development process to confirm that the organoids are displaying morphology and expressing biomarkers consistent with each development stage. Methods for imaging the organoids are known in the art, and include assessing the organoids every 2 weeks for phenotypic characterisation and visualization of 3D cell models using inverted microscopy with Extended Depth of Field (EDF), confocal imaging systems and high content analysis platforms to image and analyse the 3D inner ear organoids.

Ensuring cells are maintaining the physiological morphology, expressing markers and displaying activity expected at each developmental stage is important to ensure the quality of the organoids. Methods of measuring quality are known in the art. For example, Cell ROX® can be used to mark nuclei undergoing oxidative stress in red, and live-cell nuclei are stained blue. The cells undergoing oxidative stress (that is, those cells undergoing cell death) are not selected for further maturation. Assays of cell cytotoxicity can also be used. For example, every 2 weeks during culture, cell viability in the inner ear organoids can be assayed by Cell ROX® (CAT C10444 Thermofisher) and live/dead viability/cytotoxicity assays to evaluate the 3D cell models (CAT L7013 Thermofisher). Using such assays, organoids with viability and no cytotoxicity are selected.

As described, the development of the inner ear organoid can be tracked by testing for specific cell markers after seeding, and the organoids expressing the specific cell markers are selected for progressing to the next step. Ectodermal cell markers E-cadherin and N-cadherin can be found after about 6 days at the end of step AA1; Otic cell marker PAX8 and Otic vesicle marker PAX2 can be found after about twelve days during step AA4. The inner ear progenitor cell marker SOX2 can be found after about 18 days (from the commencement of AA2) at the end of step AA4. Hair cell markers—MyoVIIa and MyoVI typically begin to show after 33 days (from the commencement of AA2) for hair cell differentiation in step C. Inner ear neuronal markers—Tuj1 and Phalloidin can be found after about day 33 in step C.

A range of inner ear specific markers can be used to determine the growth and health of the inner ear organoids by gene expression analysis with quantitative real-time polymerase chain reaction (qRT-pCR). The organoids with inner ear specific gene expression are selected for further culture for maturation.

The cellular composition of organoids can be studied at the systems level with advances in functional genomics, including single-cell analysis and high-throughput transcriptomics to provide a more complete understanding of the development and cellular composition of inner ear organoids. These techniques will be known to those in the art.

### Compositions

In another aspect, the invention comprises a composition comprising the inner ear hair cells, or organoids comprising the inner ear hair cells formed by the methods of this invention.

Compositions of the invention may be combined with various other components to produce different therapeutic forms of the invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. See, e.g., Remington's Pharmaceutical Sciences, 19th Ed. (1995, Mack Publishing Co., Easton, Pa.) which is herein incorporated by reference.

The preferred form of the pharmaceutical composition depends on the intended mode of administration and therapeutic application. Pharmaceutical compositions prepared according to the invention may be administered by any means that leads to the composition of the invention coming into contact with the inner ear of the subject.

The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the peptide. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Additionally, auxiliary substances, such as wetting or emulsifying agents, surfactants, pH buffering substances and the like can be present in compositions. Other components of pharmaceutical compositions are those of animal, vegetable, or synthetic origin oils, for example, peanut oil, soybean oil, and mineral oil. In general, glycols such as propylene glycol or polyethylene glycol are preferred liquid carriers, particularly for injectable solutions.

The routes of administration described herein are intended only as a guide since a skilled practitioner will be able to determine readily the optimum route of administration for any particular patient.

### Methods of Treatment and Use

In yet another aspect, the present invention provides a method for treating sensorineural hearing loss in a subject in need thereof, said method comprising the step of: administering to a patient an effective amount of a composition comprising inner ear hair cells produced by the methods of the invention.

As used herein the term “patient” generally includes mammals such as: humans; farm animals such as sheep, goats, pigs, cows, horses, llamas; companion animals such as dogs and cats; primates and birds. Preferably, the patient is a human.

Sensorineural hearing loss can be diagnosed in a subject through a number of tests known in the art. For example, pure tone audiometry, which identifies hearing threshold levels of a subject, can be used to diagnose sensorineural hearing loss. Other tests that can be used to diagnose, and/or measure any improvement or deterioration of sensorineural hearing loss include the otoacoustic emissions test and the auditory brainstem response test.

The compositions of the invention can be used in regenerative medicine applications, whereby the patient's damaged tissue is replaced or regenerated. In an embodiment, the compositions of the invention may be transplanted into a patient in need thereof. For example, the inner ear hair cells and organoids produced by the methods of this invention can be directly transplanted into a patient in need thereof. In a further example, inner ear organoids comprising inner ear hair cells can be produced by the methods of the invention using the patient's own iPSCs. Once inner ear hair cells have been generated, these cells can be delivered back into the patient's cochlea in position for development and application. Known transplantation techniques in the art can be used to deliver the compositions of the invention comprising inner ear hair cells into the cochlea of a patient.

Alternatively, the compositions of the invention can be used in cell therapy, bioprinting and tissue engineering applications. For example, 3D bioprinting can be used to bioprint nanoparticle material with patient derived inner ear hair cells and organoids produced by the methods of the invention into specific shapes and delivering the repaired hair cells back into the patient cochlea for therapeutic development and application.

In therapeutic applications, pharmaceutical compositions or medicaments are administered to a patient suspected of, or already suffering from, such a disease in an amount sufficient to at least partially arrest the symptoms of the disease and its complications. An amount adequate to accomplish this is defined as a therapeutically- or pharmaceutically effective dose.

In another aspect of the invention, there is provided a method of treating sensorineural hearing loss in a subject in need thereof comprising the step of administering a SHH inhibitor to the subject's inner ear.

In further aspect of the invention, there is provided a method of regenerating cells in the inner ear of a subject comprising the step of administering a SHH inhibitor to the subject's inner ear. Preferably, the cells in the inner ear are selected from inner ear hair cells, or supporting cells of the cochlea or vestibular system.

In some embodiments of this aspect of the invention, the subject may be suffering from sensorineural hearing loss. In other embodiments, the subject may have a disorder of the vestibular system.

Preferably, the SHH inhibitor is selected from the list of cyclopamine (SMO inhibitor), GANT58 or GANT61 (GLI inhibitor).

The SHH inhibitor is administered in an amount to partially inhibit the activity of the SHH pathway. Preferably, the SHH inhibitor administered at a concentration of 0.5 to 20 μM e.g., 0.5, 1 μM, 1.5 μM, 2 μM, 2.5 μM, 3 μM, 4 μM, 5 μM, 7 μM, 8 μM, 9 μM, 10 μM, 11 μM, 12 μM, 13 μM, 14 μM, 15 μM, 16 μM, 17 μM, 18 μM, 19 μM, 20 μM or another concentration from about 0.5 μM to about 20 μM. Most preferably, the SHH inhibitor is administered at a dose of 0.5 mg/Kg to 20 mg/Kg of the subject, once per day.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human, but in some embodiments, the patient can be an animal exhibiting sensorineural hearing loss. Treatment dosages need to be titrated to optimize safety and efficacy.

Preferably, a sufficient number of organoids comprising inner ear hair cells produced by the methods of the invention are implanted in the subject to treat sensorineural hearing loss. Preferably between 1 to 1000 organoids are transplanted into a subject in need of treatment. Most preferably, between 1 to 100 organoids generated by methods of the invention using each of cyclopamine, GANT58 and GANT61 are transplanted into a subject in need of treatment.

The compositions of the invention can also be used to identify optimal dosages for other therapeutic agents designed to act on the subject's inner ear. For example, therapeutic agents can be applied to a population of inner ear hair cells or an organoid comprising inner ear hair cells produced by the methods of this invention, and the optimal dose for the desired effect of the therapeutic agent can be determined.

In some embodiments of the invention, the compositions of the invention are administered together with additional therapeutic agents. For example, the compositions of the invention may be administered together with anti-inflammatory drugs, which can upregulate cytokine and ion hemostasis in the inner ear. Preferably the anti-inflammatory drugs are selected from prednisone or dexamethasone. Most preferably, the prednisone or dexamethasone is administered at a concentration of approximately 4-10 mg/mL.

The inventor has surprisingly identified that Cdo homozygous and SHH heterozygous knockout mice demonstrate increased inner hair cell differentiation compared with wild-type mice, suggesting a possible mechanism of action for how SHH inhibitors may act to increase inner ear hair cell differentiation in the present invention. The inventor has also identified that Cdo homozygous and SHH heterozygous knockout mice exhibiting increased hair cell differentiation show inhibition of SHH pathway between 50% to 70%.

Accordingly, in a further embodiment of the invention, there is provided a method of enhancing inner ear hair cell differentiation in a subject comprising the step of partially inhibiting Cdo expression in the subject. Preferably, Cdo expression is inhibited by the administration of siRNA or CRISPR in a therapeutically effective amount.

In a further embodiment of the invention, there is provided a method of increasing the number of inner ear hair cells in a subject comprising the step of partially inhibiting Cdo expression in the subject. Preferably, Cdo expression is inhibited by the administration of siRNA or CRISPR in a therapeutically effective amount.

In a further embodiment of the invention, there is provided a method of increasing the number of inner ear hair cells in a subject comprising the step of partially inhibiting the SHH pathway by inhibiting Cdo in the subject. Preferably, Cdo is inhibited by the administration of siRNA or CRISPR in a therapeutically effective amount.

In a further embodiment of the invention, there is provided a method of increasing the number of inner ear hair cells in a subject comprising the step of partially inhibiting Cdo in the subject. Preferably, Cdo is inhibited by the administration of siRNA or CRISPR in a therapeutically effective amount.

In another embodiment, the invention comprises the use of the compositions of the invention in the manufacture of a medicament for the treatment of sensorineural hearing loss in a subject in need thereof.

Modifications of the above-described methods will be apparent to those skilled in the art. The above embodiments of the invention are merely exemplary and should not be construed to be in any way limiting.

### Screening Methods and Models

In yet another aspect of the invention, the inner ear hair cells and organoids produced by the methods of the invention can be used to assess the ototoxicity of a test agent. In another aspect, the inner ear hair cells and organoids produced by the methods of the invention can also be used to assess the safety and efficacy of therapeutic compounds that are designed to target inner ear hair cells. Patient-derived inner ear hair cells and organoids produced by the methods of the invention using patient specific iPSCs or from adult stem or progenitor cells can serve as patient-specific clinical models for drug screening, or models for diagnosing patient specific conditions.

The inner ear hair cells and organoids comprising the inner ear hair cells produced by the methods of this invention are capable of simulating biological tissues, in a manner similar to the living body. In an aspect, there is provided a method for assessing the ototoxicity or therapeutic effectiveness of a test agent comprising a step of treating with a test agent a population of inner ear hair cells or an organoid comprising inner ear hair cells produced by the methods of this invention.

The test agent may be selected from any bioactive substances and may be selected from the group consisting of small molecular chemicals, peptides, proteins (for example, antibodies, or other protein drugs), or nucleic acid molecules or extracts (for example, animal or plant extracts). The method may be for screening a test agent having a therapeutic effect on an otological disease, such as sensorineural hearing loss. Alternatively, the method may be for screening a test agent having some other therapeutic effect to assess its ototoxicity.

The effect of the test agent on ototoxicity or the therapeutic effectiveness of the agent can be assessed using known methods. In one embodiment an organoid or inner ear hair cell population generated according to the methods of this invention is treated with a test agent. The viability of the organoid or inner ear hair cells is compared to the viability of an untreated control organoid or inner ear hair cell population to characterize the toxicity or therapeutic effectiveness of the candidate compound. Alternatively, the control population can be an organoid or inner ear hair cell population treated with a test agent with a known level of toxicity or therapeutic effect.

The inner ear hair cells and organoids as described herein can be used as a clinical model for deafness and can be used to study the role of specific genetic markers in deafness. Patient-derived organoids from iPSCs or from adult stem or progenitor cells can serve as patient-specific clinical models for drug screening, as well as a diagnostic tool. In an aspect, there is provided a method of diagnosing an otological disease in a patient comprising the step of evaluating for the presence or absence of a marker specific for the disease in a population of patient derived inner ear hair cells or a patient derived organoid comprising inner ear hair cells produced by the methods of this invention. Preferably, the marker is a genetic marker associated with hearing loss. Most preferably, the marker is chosen from the list of GJB2, STRC, OTOF, SLC26A4, MYO7A, TECTA, MYO15A, CDH23, USH2A and WFS1.

### EXAMPLES

### Example 1: Protocol Using Exemplar Agent to Promote Hair Cell Differentiation of Human iPS Cells

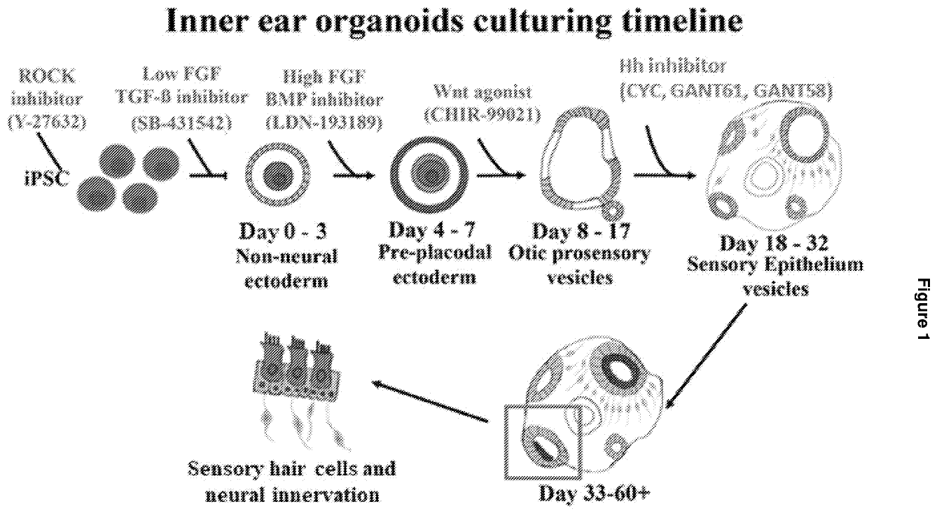

FIG. 1 illustrates methods for generating inner ear hair cells from the induced pluripotent stem cells. The inventor added a combination of agents and inhibitors at developmental stages of the inner ear organoid culture to produce the invention. In particular:
(i) ROCK inhibitor (Y-27632) is added to iPSC in suspension culture to promote the formation of three-dimensional embryoid bodies for about 3 days.
(ii) TGF-β inhibitor (SB-431542) is added at low concentration of FGF to drive the development of non-neural ectoderm in days 0 to 3.
(iii) The cultures are then transitioned to medium containing a high concentration of FGF and BMP inhibitor (LDN-193189) during days 4-7, which drives development of the early pre-otic placodal epithelium/pre-placodal ectoderm.
(iv) Next, Wnt agonist (CHIR-99021) is added to stimulate development of otic placode from days 8-11, otic pit formation from days 12-14 and otic prosensory vesicles from days 15-17.
(v) From days 18-32, hedgehog signalling inhibitor (CYC, GANT58 or GANT61) is added to promote the formation of sensory epithelium vesicles, and continued culture under these conditions results in the formation of sensory hair cells with neural innervation from day 33 onwards.

### Example 2: Application of Protocol of Example 1

(i) Formation of Embryoid Bodies

Human induced pluripotent stem cells of the cell line Gibco Human Episomal iPSC line, Thermo Fisher A18945 were cultured in a 6 well suspension plate in mTeSR™1® medium (STEM CELL Technologies) with ROCK inhibitor (CAT Y-27632 STEM CELL Technologies, final concentration of 10 μM) to maintain the Human Pluripotent Stem Cells and form human embryoid bodies for 2 days.

(ii) Formation of Non-Neural Ectoderm

The embryoid bodies from (i) were transferred to a 6-well suspension culture plate containing chemically defined medium on day 0. The chemically defined medium contained the reagents in Table 1:

TABLE 1

| | | | Catalogue | Company |
|---|---|---|---|---|
| Reagents | Volume | | Number | Name |
| Hams F12 GlutaMax | 21.75 | mL | 31765-035 | Gibco |
| Iscove's Modified Dulbecco's | 21.75 | mL | 31980-030 | Gibco |
| Medium (IMDM) GlutaMax medium | | | | |
| 0.5% Bovine Serum Albumin (BSA) | 5 | mL | A2153 | Sigma |
| Chemically defined lipid concentrate | 500 | μL | 11905031 | Gibco |
| Insulin solution human | 35 | μL | I9278 | Sigma |
| Transferrin | 500 | μL | T3309 | Sigma |
| α-Monothioglycerol | 2 | μL | M6145 | Sigma |
| Penicillin Streptomycin | 500 | μL | 15070-063 | Gibco |

Upon embryoid body formation on day 0, Fibroblast growth factor 2 (FGF2) (CAT 78003.2 from STEMCELL Technologies) was added to the chemically defined medium to reach a final concentration of 4 ng/mL. The TGFβ inhibitor SB-431542 (CAT 72232 from STEMCELL Technologies) was also added to the chemically defined medium to reach a final concentration of 10 μM.

The cells were cultured until 3 for non-neural ectoderm formation. The presence of non-neural ectoderm was confirmed by immunostaining and qRT-PCR analyses.

(iii) Formation of Otic Pro-Sensory Vesicles

On day 4, FGF2 (CAT 78003.2 from STEMCELL Technologies) was added to the cell culture medium to reach a final concentration of 50 ng/mL. The BMP inhibitor LDN-193189 (CAT 72146 from STEMCELL Technologies) was also added to reach a final concentration of 200 nM. The organoids were cultured in 6-well suspension culture plate for pre-otic placodal epithelium formation until day 7.

On day 8, the WNT agonist CHIR-99021 (CAT 72052 from STEMCELL Technologies) was added to the organoids embedded in 10% Matrigel® to reach a final concentration of 3 μM. The organoids were cultured in a 6-well suspension culture plate for otic placode formation.

On day 12, 1-6 organoids were transferred into each well of a 48 well suspension plate (GBO, 677102), by resuspending organoids in organoid maturation medium with 10% Matrigel® plus CHIR99021. The medium was replaced after incubating the organoids for an hour, to allow the Matrigel® to set.

From days 12-17, CHIR-99021 was added to the culture to maintain a final concentration of 3 μM and enable otic pit formation. Otic prosensory vesicles were observed on day 17. The presence of otic prosensory vesicles was confirmed by immunostaining and confocal imaging.

(iv) Formation of Inner Ear Hair Cells by Addition of Cyclopamine

On day 18, the SHH inhibitor cyclopamine (CAT 239803 from Merck Millipore) was added to the cell culture to reach a final concentration of 1 μM. Matrigel® was also added to the cell culture to reach a concentration of 10%. The cells were cultured in organoid maturation medium until day 33.

On day 33, the cyclopamine was removed from the medium by washing and the droplet aggregates were remained in the cell culture containing 10% Matrigel®.

The cells were then cultured for up to 200 days in the Organoid Maturation Medium containing 10% Matrigel®. 200 μL of medium was changed daily for each well. The cells were cultured until 60-100 days, until an examination of the cell's morphology determined maturation. The viable, medium size and round 3D shape organoids with inner ear sensory cell gene expression markers were selected for analysis.

Inner ear hair cells and nerve innervation was detected after Day 35. The presence and number of inner ear hair cells was detected by immunostaining and capture with confocal imaging.

### Example 3: Alternative Application of Protocol of Example 1

(v) Formation of Inner Ear Hair Cells by Addition of GANT58

Steps (i) to (iii) were followed as per Example 2.

On day 18, the SHH inhibitor GANT58 (CAT 73984 from STEM CELL Technologies) was added to the cell culture to reach a final concentration of 1 μM. Matrigel® was also added to the cell culture to reach a concentration of 10%. The cells were cultured in organoid maturation medium until day 33.

On day 33, the GANT58 was removed from the medium by washing and the droplet aggregates were remained in the cell culture containing 10% Matrigel®.

The cells were then cultured for up to 200 days in the Organoid Maturation Medium containing 10% Matrigel®. 200 μL of medium was changed daily for each well. The cells were cultured until 60-100 days, until an examination of the cell's morphology determined maturation. The viable, medium size and round 3D shape organoids with inner ear sensory cell gene expression markers were selected for analysis.

Inner ear hair cells and nerve innervation was detected after Day 35. The presence and number of inner ear hair cells was detected by immunostaining and capture with confocal imaging.

### Example 4: Alternative Application of Protocol of Example 1

(vi) Formation of Inner Ear Hair Cells by Addition of GANT61

Steps (i) to (iii) were followed as per Example 2.

On day 18, the SHH inhibitor GANT61 (CAT 73692 from STEMCELL Technologies) was added to the cell culture to reach a final concentration of 1 μM. Matrigel® was also added to the cell culture to reach a concentration of 10%. The cells were cultured in organoid maturation medium until day 33.

On day 33, the GANT61 was removed from the medium by washing, and the droplet aggregates were remained in the cell culture containing 10% Matrigel®.

The cells were then cultured for up to 200 days in the Organoid Maturation Medium containing 10% Matrigel®. 200 μL of medium was changed daily for each well and cultured for day 60-100, until an examination of the cell's morphology determined maturation. The viable, medium size and round 3D shape organoids with inner ear sensory cell gene expression markers were selected for analysis.

Inner ear hair cells and nerve innervation was detected after Day 35. The results of inner ear hair cells were detected by immunostaining and capture with confocal imaging.

### Example 5: The Effect of the Cdo Gene in Inner Ear Hair Cell Differentiation

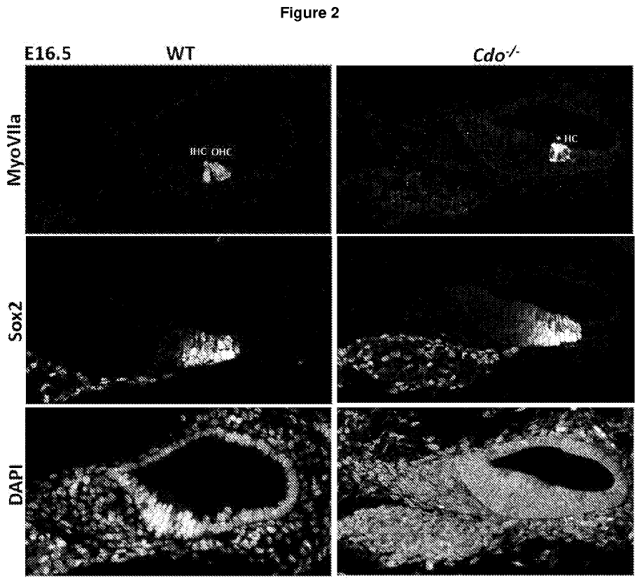

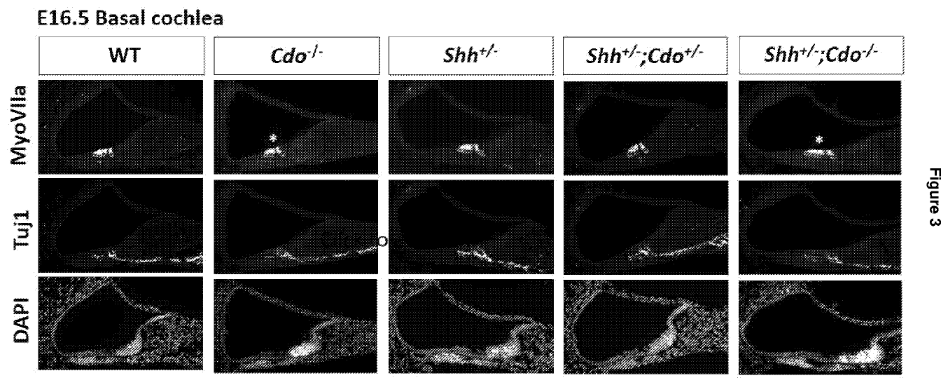

To show the effect of the Cdo gene in inner ear hair cell differentiation, the inventor performed immunohistochemistry of tissue sections from the inner ear Organ of Corti from mice at developmental stage E16.5. In this respect FIG. 2 illustrates the extra formation of hair cells and expansion of supporting cells marker by using hair cell marker MyosinVIIa and supporting cell marker Sox2 in the Organ of Corti of E16.5 Cdo^{-/-} mutants in 10× magnification.

In FIG. 2, cochlear hair cells are labelled using the hair cell-specific marker MyosinVIIa (MyoVIIa) and supporting cells are labelled using the supporting cell-specific marker Sox2. The nuclei of all cells are counterstained with DAPI.

The three images on the left are from wildtype (WT) mice that have normal hearing, whilst the three images on the right are from Cdo^{-/-} homozygous knockout mice that have profound hearing loss.

Knockout of both copies of the Cdo gene in mice results in an expansion of the supporting cell population at E16.5 as indicated by the increase in Sox2 positive cells in the Cdo^{-/-} mice.

### Example 6: The Effect of SHH Signalling on Cdo Function in Inner Ear Hair Cell Differentiation

To show the effect of SHH signalling on Cdo function in inner ear hair cell differentiation, immunohistochemistry of tissue sections from the inner ear Organ of Corti from mice at developmental stage E16.5 was performed. The inventor divided the cochlea into three different regions of the cochlear spiral: basal, medial, and apical, as indicated.

FIG. 3 illustrates supernumerary hair cells in Shh^{+/-}Cdo^{-/-} compound mutant cochlea using hair cell marker MyosinVIIa and neural marker beta-tubulin III Tuj1 antibodies to mark the hair cells and nerve innervation into cochlea at E16.5 in 10× magnification. Cochlear hair cells labelled using the hair cell-specific marker Myosin VIIa (MyoVIIa) and neurons are shown labelled using the neuron-specific marker Beta-tubulin III, clone TUJ1 (Tuj1). The nuclei of all cells are counterstained with DAPI.

A total of five different mouse models are shown in FIG. 3:
1. wildtype mice (WT);
2. Cdo homozygous knockout mice (Cdo^{-/-}) that are missing both copies of the Cdo gene;
3. Shh heterozygous mice (Shh^{+/-}) that express only one copy of the Shh gene;
4. Shh and Cdo compound heterozygous mice (Shh^{+/-}; Cdo^{+/-}) that express only one copy of each gene; and
5. Shh heterozygous Cdo homozygous knockout compound mutant mice (Shh^{+/-}; Cdo^{-/-}) that express one copy of the Shh gene and are missing both copies of the Cdo gene.

Shh^{+/-}; Cdo^{-/-} compound mutant mice have a significantly increased number of cochlear hair cells and restored innervation of these hair cells.

### Example 7: Quantification of the Number of Ectopic Hair Cells with Nerve Innervation Formed in Shh^{+/-}; Cdo^{-/-} Compound Mutant Cochlea

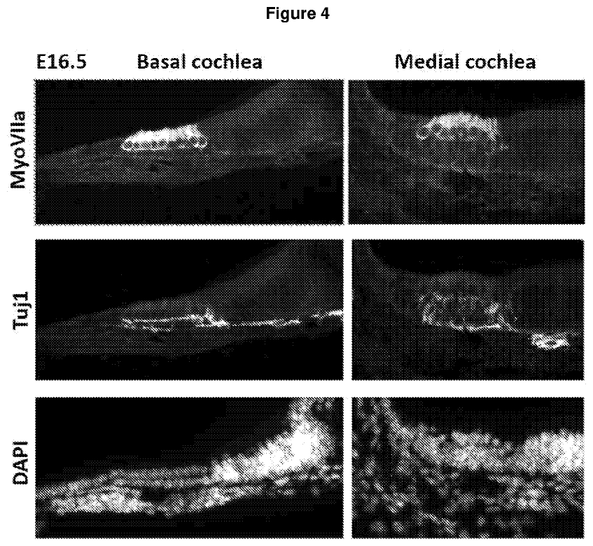

To quantitate the number of ectopic hair cells with nerve innervation formed in Shh^{+/-}; Cdo^{-/-} compound mutant cochlea, the inventor performed a cell counting analysis using ImageJ software of tissue section immunohistochemistry from the inner ear Organ of Corti dissected from mice at developmental stage E16.5 at the basal and medial regions of the cochlea. FIG. 4 illustrates ectopic hair cells with nerve innervation in Shh^{+/-}; Cdo^{-/-} compound mutant cochlea using hair cell marker Myosin VIIa and neural marker Tuj1 antibodies to mark the hair cells and nerve innervation into E16.5 cochlea in 20× magnification. Cochlear hair cells are labelled using the hair cell-specific marker Myosin VIIa (MyoVIIa) and neurons are labelled using the neuron-specific marker Beta-tubulin III, clone TUJ1 (Tuj1). The nuclei of all cells are counterstained with DAPI.

### Example 8: Change of Pillar Cells Supporting Cells in the Cdo^{-/-} Mutants

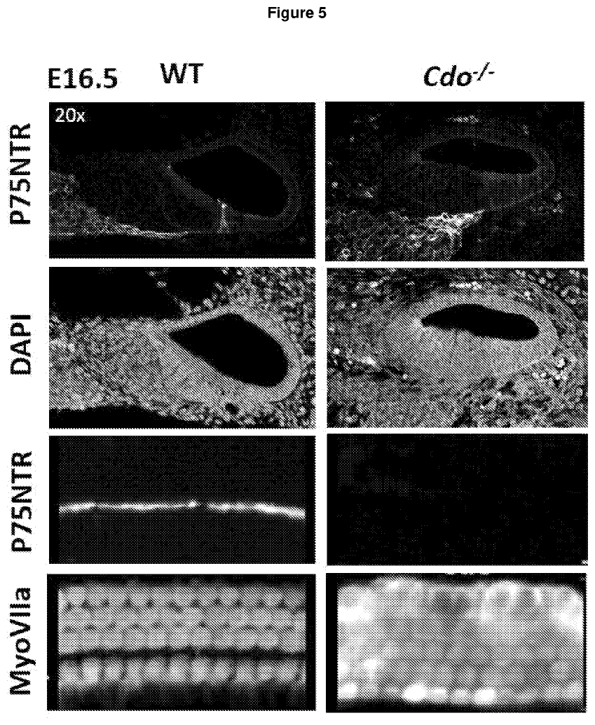

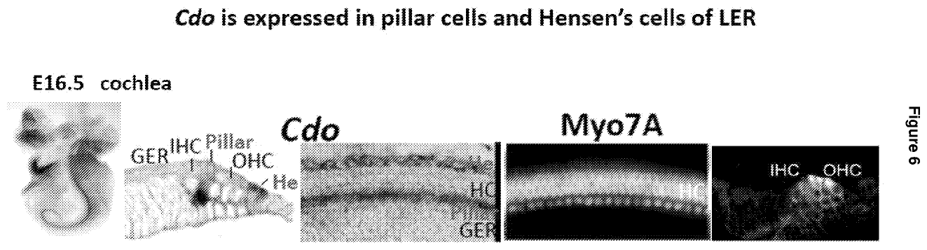

To observe any change of Pillar cells supporting cells in the Cdo/mutants, the inventor performed immunostaining on sections and whole-mount of cochlear tissue from wildtype (WT) and Cdo knockout (Cdo^{-/-}) mice at embryonic day 16.5 with a Pillar cell-specific marker P75NTR. Cell nuclei have been counterstained with DAPI and images have been captured at 20× magnification. FIG. 5 illustrates missing of Pillar cells in Cdo^{-/-} mutants. The expression and distribution of Pillar cells in the inner ear is clearly disrupted in the Cdo knockout cochlea. The Pillar cells that normally reside between the inner and outer hair cells in the wildtype are absent in the knockout mice.

### Example 9: Cdo Expression in Supporting Cells in Mouse Cochlea at E16.5

The Organ of Corti derives from a prosensory domain that runs the length of the cochlear duct and is bounded by two nonsensory domains, Kolliker's organ on the neural side Greater Epithelial Ridge (GER) and the outer sulcus on the abneural side Lesser Epithelial Ridge (LER). The mechanisms that establish sensory and nonsensory territories in the cochlea duct is not clearly known, but Shh signalling is likely to play a significant role.

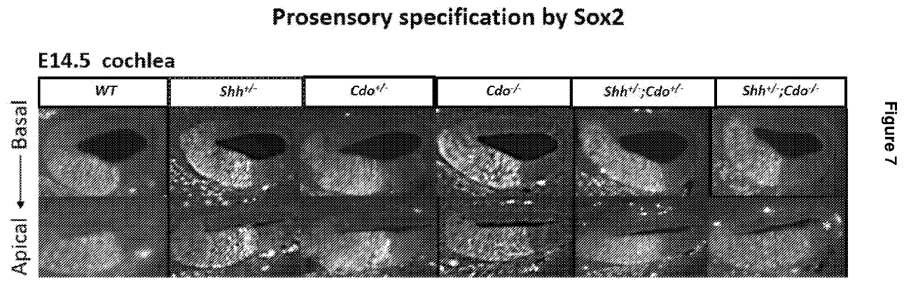

To investigate whether Cdo is involved in the specification of nonsensory cell types, the expression of Cdo gene in the otic epithelium was examined. FIG. 6 illustrates Cdo expression in supporting cells in mouse cochlea at E16.5. By RNA in situ hybridisation, it was observed that Cdo is expressed specifically in the Hensen cells of cochlea at E16.5, it is not expressed in the sensory hair cells which are marked by wholemount MyoVIIa staining. Cdo displays differential expression in the nonsensory epithelium and specifically in the Hensen cells and pillar cells. The expression of Cdo in the nonsensory domains suggest that it may be required to maintain the nonsensory cell fates and/or involved in regulating the differentiation of the organ of Corti.

To identify the Cdo expression profile in inner ear cochlea, transverse and longitudinal section and whole-mounts of wildtype mouse cochlea sections from developmental day E16.5 were shown. In FIG. 6, brightfield images show Cdo expression (dark) and fluorescent images show immunolabelling with the hair cell-specific marker MyosinVIIa (Myo7A). Labels indicate the position of the Greater Epithelial Ridge (GER), inner hair cells (IHC), outer hair cells (OHC), and Lesser Epithelial Ridge (LER). The overlay indicates the location of Cdo expression is not expressed in relative to the inner ear and outer hair cells.

### Example 10: Prosensory Domain Specification in Cdo and Cdo/Shh Compound Mutants

To observe any change in early prosensory domain specification, immunostaining on sections taken at different regions of the cochlea from wildtype (WT) and a series of mutant mice at embryonic day 14.5 was performed. Immunostaining with Sox2 antibody was performed to indicate the prosensory domain. FIG. 7 illustrates prosensory domain specification in Cdo and Cdo/Shh compound mutants. A total of six different mouse models are shown here:
1. wildtype mice (WT);
2. Shh heterozygous mice (Shh^{+/-}) that express only one copy of the Shh gene;
3. Cdo heterozygous mice (Cdo^{+/-}) that express only one copy of the Cdo gene;
4. Cdo knockout mice (Cdo^{-/-}) that are missing both copies of Cdo;
5. Shh and Cdo compound heterozygous mice (Shh^{+/-}; Cdo^{+/-}) that express only one copy of each gene; and
6. Shh heterozygous Cdo homozygous knockout compound mutant mice (Shh^{+/-}; Cdo^{-/-}) that express one copy of the Shh gene and are missing both copies of the Cdo gene.

### Example 11: Cell Cycle Exit by P27Kip1 in Cdo and Cdo/Shh Compound Mutants

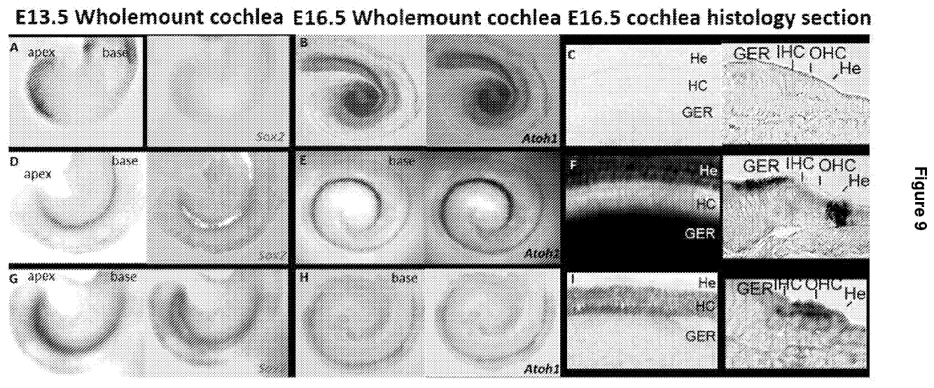

To observe any change in cell cycle exit in epithelium of cochlea at E14.5, immunostaining was performed on sections taken at different regions of the cochlea from wildtype (WT) and a series of mutant mice at embryonic day 14.5. Immunostaining was performed with P27^{Kip1} antibody to indicate the prosensory domain specification. FIG. 8 illustrates cell cycle exit by P27Kip1 in Cdo and Cdo/Shh compound mutants. A total of six different mouse models are shown here:
1. wildtype mice (WT);
2. Shh heterozygous mice (Shh^{+/-}) that express only one copy of the Shh gene;
3. Cdo heterozygous mice (Cdo^{+/-}) that express only one copy of the Cdo gene;
4. Cdo knockout mice (Cdo^{-/-}) that are missing both copies of Cdo;
5. Shh and Cdo compound heterozygous mice (Shh^{+/-}; Cdo^{+/-}) that express only one copy of each gene; and
6. Shh heterozygous Cdo homozygous knockout compound mutant mice (Shh^{+/-}; Cdo^{-/-}) that express one copy of the Shh gene and are missing both copies of the Cdo gene.

The data showed premature cell cycle exit in epithelium of cochlea in Cdo/Shh compound mutants, as shown in FIG. 8.

### Example 12: Gli Gene Expression in SHH Pathway in Mouse Cochlea at E13.5 and E16.5

To identify the expression pattern of Gli1, Gli2 and Gli3 genes in SHH signalling pathway in mouse cochlea at E13.5 and E16.5, the inventor performed RNA in situ hybridization and immunostaining on whole-mount wildtype cochlea. FIG. 9 illustrates Gli gene expression in SHH pathway in mouse cochlea at E13.5 and E16.5. This panel of images are cochlea tissue sections from wildtype mice taken at different stages of development and stained to show the expression of Gli1, Gli2 and Gli3 throughout the development of the embryo at E13.5 for prosensory specification stage and E16.5 for hair cell differentiation stage.

Gli1 and Gli2 are the readout for Hh signalling. Gli3 is known to be the repressor of Hh signalling. At E13.5, Sox2 is expressed in prosensory domain shown in dark. Gli2 and Gli3 are co-expressed with Sox2 in prosensory domain, however, Gli1 is not detected in the prosensory epithelium region. Importantly, Gli2 and Gli3 expression in the cochlea region are detected throughout the development of cochlea at E13.5-16.5.

At E16.5, Atoh1 is expressed in cochlear hair cells at E16.5. Gli1 is expressed in spiral ganglion in cochlea. Importantly, Gli2 as a SHH activator is not expressed in sensory hair cells but restricted in Greater Epithelial Ridge (GER) and Hensen cells (He) which is the reciprocal expression pattern to Atoh1. Gli3 as a SHH repressor is expressed specifically in sensory hair cells overlapping to Atoh1 expression.

### Example 13: Gross Morphology of Human iPSCs Derived Inner Ear Organoid at Day 0-10

To assess the gross morphology of organoid cultures over time Day 0-10, per the protocol described in Example 1, the size and morphology of organoids at different stages was observed by using phase contrast microscopy.

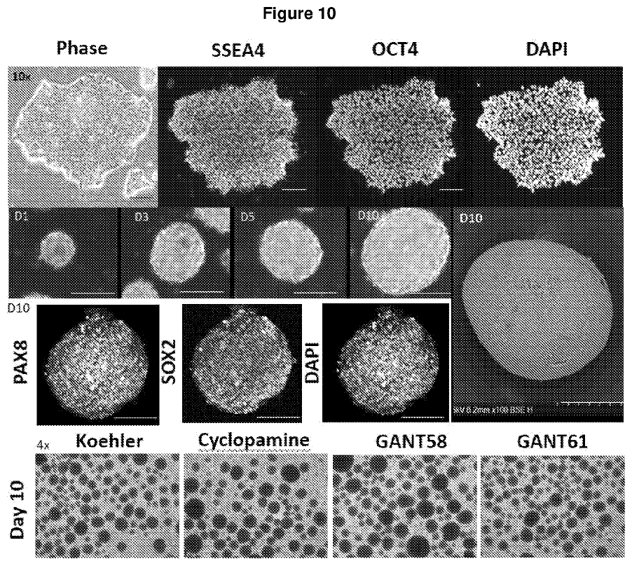

FIG. 10 illustrates embryoid body formation at day 0-5, the aggregates were three-dimensional spherical structure. At Day 10, immunostaining was performed on whole-mount inner ear organoid from normal GIBCO cells. Immunostaining with PAX8 and SOX2 antibodies was performed to indicate the early otic identity.

### Example 14: Human iPSCs Derived Inner Ear Organoid at Day 20-40—Ectodermal Cell Fate

The gross morphology of Human iPSCs derived inner ear organoids that were developed using:
(a) the method described in Koehler et. al. (2017);
(b) the method described in Example 1, using cyclopamine as the SHH inhibitor in step (v);
(c) the method described in Example 1, using GANT58 as the SHH inhibitor in step (v) (Example 3); and
(d) the method described in Example 1, using GANT61 as the SHH inhibitor in step (v). were compared.

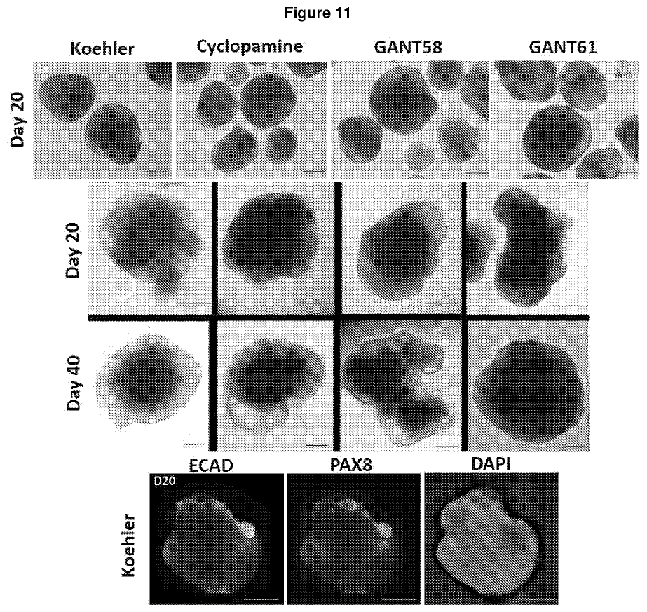

FIG. 11 illustrates gross morphology of Human iPSCs derived inner ear organoid at day 20-40 in 10× magnification. Characterization of inner ear organoid at Day 20 by using otic PAX8 and ECAD positive epithelium bore a morphological resemblance to the developing otic structures. That the epithelium re-organization is clearly visible through the aggregate surface. FIG. 11 shows that the efficiency of the cellular re-organization is about 90-95% of aggregates using the Koehler method. However, the method of Example 1 using cyclopamine, GANT58 or GANT61 demonstrated higher efficiency of cellular re-organization than Koehler method with about 95-100% of aggregates.

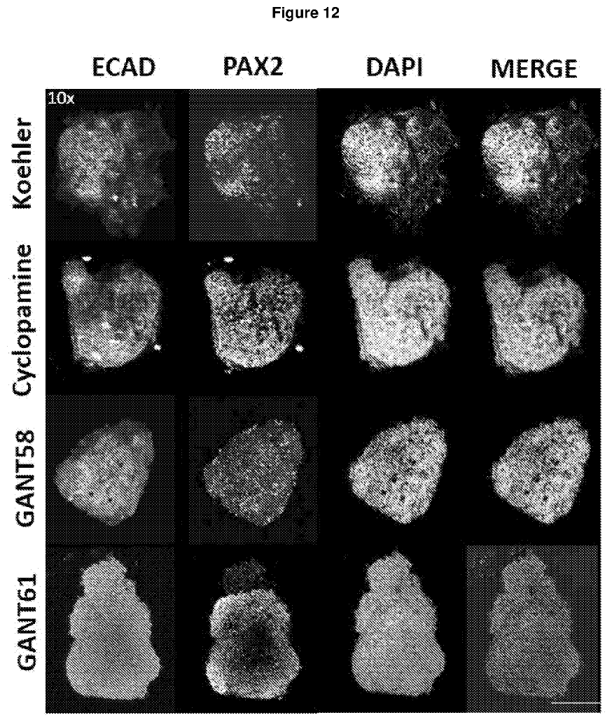

To identify the ectodermal cell fate of inner ear organoids at Day 20-40, the ECAD and PAX2 expression in organoids was observed by using confocal microscopy, comparing organoids developed using (a) Koehler's method without Hh inhibitors, (b) the method of Example 1 with cyclopamine as the SHH inhibitor (Example 2), (c) the method of Example 1 with GANT58 as the SHH inhibitor (Example 3) and (d) the method of Example 1 with GANT61 as the SHH inhibitor (Example 4). E-CADHERIN and PAX2 stained organoids showed ectodermal cell fate. The ectodermal cell fate of the inner ear organoids developed better with the SHH inhibitors compared with the Koehler method. FIG. 12 shows that organoids that were developed using the method of Example 1 including Hh inhibitors in step (v) showed a greater number of cells expressing the ectodermal cell fate as compared with the Koehler method. The formation of ectodermal cell appeared to be continuous, beginning on day 7 until approximately day 20-40. On day 20, CYC, GANT58 and GANT61 treated aggregates contained a higher abundance of ECAD/PAX2 positive vesicles with a luminal diameter greater than 50 μm.

### Example 15. Human iPSCs Derived Inner Ear Organoid at Day 14-40—Otic Cell Fate

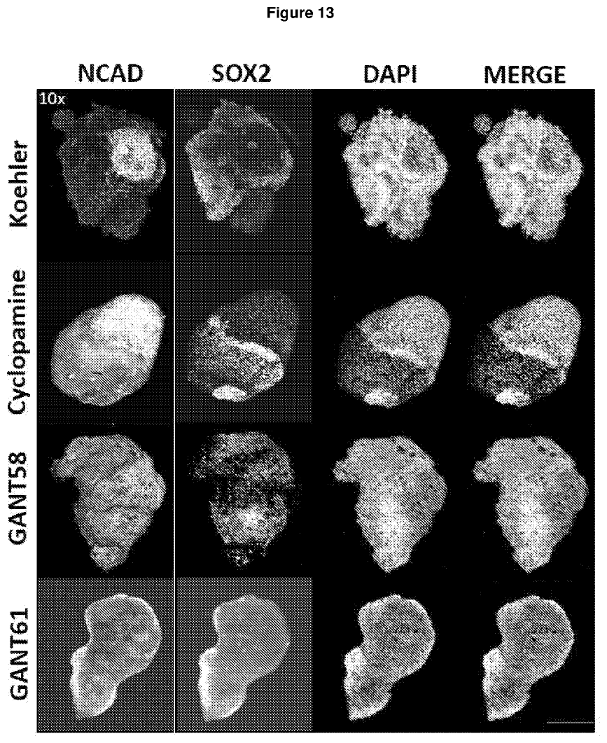

To identify the otic cell fate of inner ear organoids at Day 14-40, the NCAD and SOX2 expression in organoids by using confocal microscopy was observed. The results are presented in FIG. 13, showing the results for Koehler's method without Hh inhibitors, the method of Example 1 with cyclopamine (Example 2), the method of Example 1 with GANT58 (Example 3) and the method of Example 1 with GANT61 (Example 4). FIG. 13 shows that organoids that were developed using the method of Example 1 including Hh inhibitors in step A showed a greater number of cells expressing the otic cell fate as compared with the Koehler method. That is, the otic cell fate of inner ear organoids developed better with the SHH inhibitors when compared with the Koehler method.

### Example 16. Human iPSCs Derived Inner Ear Organoid at Day 33-60

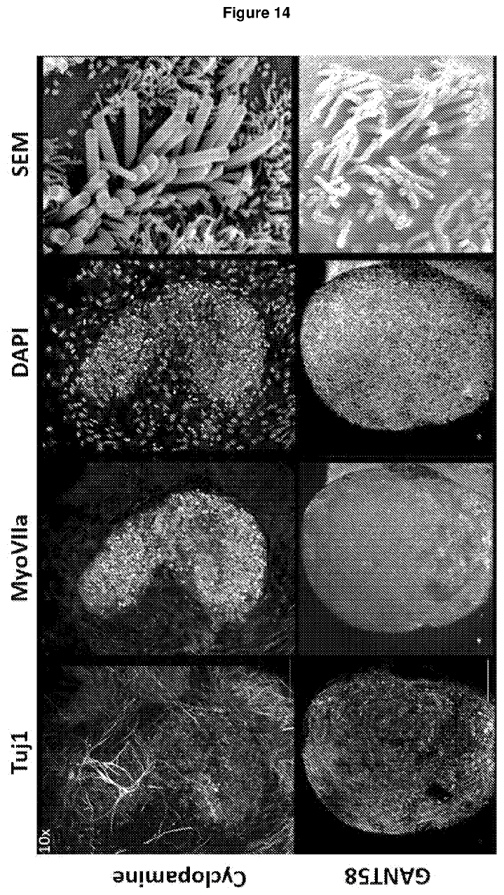

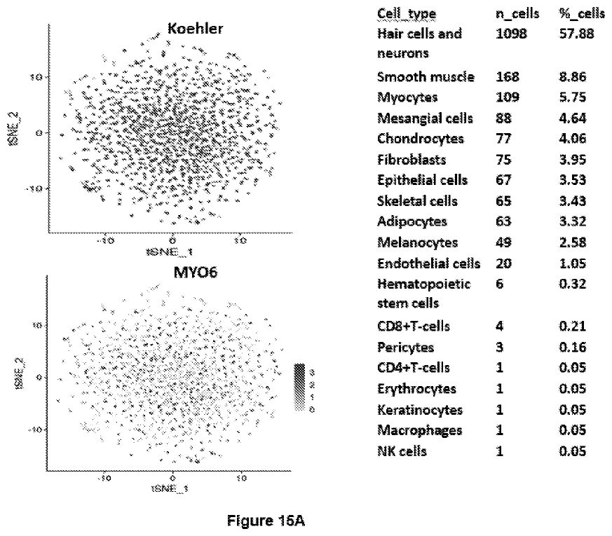

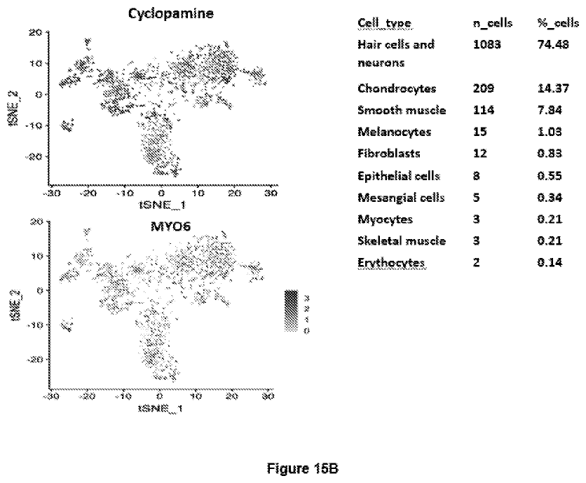

To identify the development of sensory hair cells of inner ear organoids at Day 33-60, the MyosinVIIa and Tuj1 expression in organoids was observed by using confocal microscopy. The results are presented in FIG. 14, which compares organoids developed using (a) the method of Example 1 with cyclopamine (Example 2), and (b) the method of Example 1 with GANT58 (Example 2). FIG. 14 shows that sensory hair cells and supporting cells were present in organoids developed using both SHH inhibitors. An opaque outer cell mass is indicative of complete re-organization, which was found in organoids developed using each of the SHH inhibitors. In comparison, less translucent epithelium indicative of incomplete or partial re-organization was found in organoids developed using the Koehler method. Sensory hair cell epithelium through the aggregate surface was found in organoids developed using each SHH inhibitor. These results suggest that SHH signalling inhibition can increase the number of inner ear hair cells derived from preplacodal ectoderm.

### Example 17: Cell Fate Analysis on Human iPSCs Derived Inner Ear Organoid by Single Cell RNA Sequencing

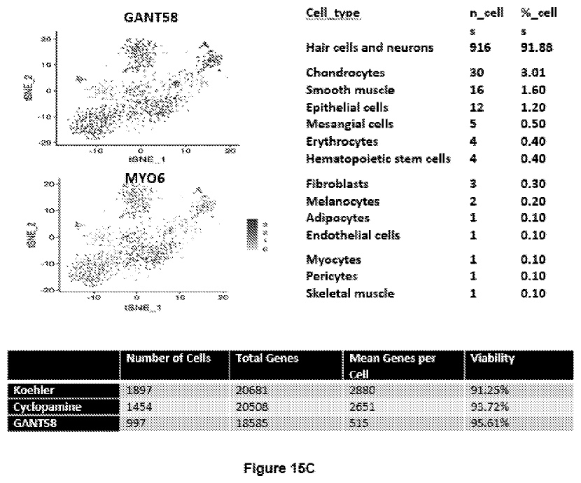

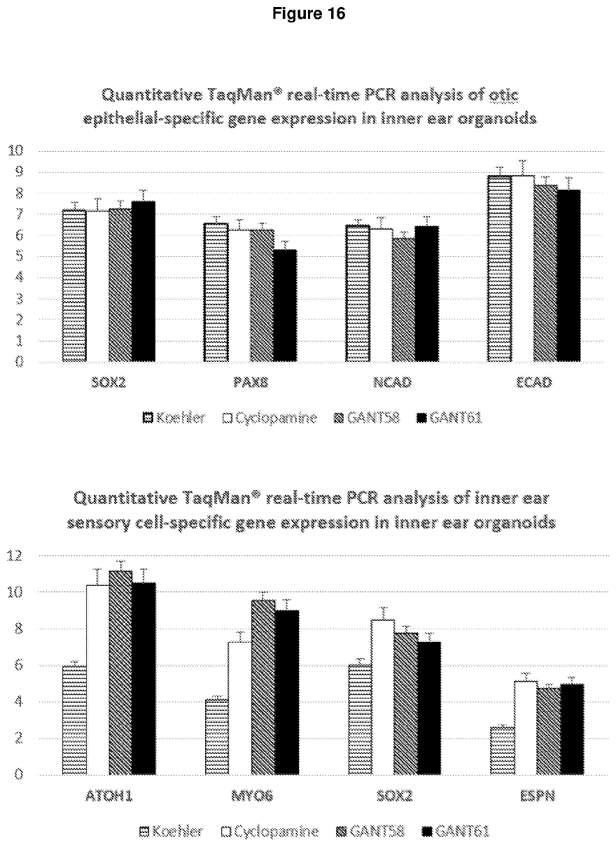

To verify the hair cell marker genes in RNAseq transcriptome analysis, quantitative TaqMan® real-time polymerase chain reaction (qRT-PCR) in inner ear organoids was performed to identify the level of hair cell gene expression. FIG. 15 illustrates sensory hair cell-specific gene expression analysis on human iPSCs derived inner ear organoid at Day 60 by single cell RNA sequencing transcriptome analysis. FIG. 16 illustrates otic epithelial-specific gene expression in inner ear organoids at Day 20 and inner ear sensory cell-specific gene expression in inner ear organoids at Day 60 by TaqMan® qRT-PCR analyses. The presence of MyoVI represents inner ear hair cell differentiation. The single cell RNA sequencing analysis identified the hair cell and neuronal markers equating to ˜91.88% in organoids developed using GANT58, ˜74.48% in organoids developed using CYC and ˜57.88% in organoids developed using the Koehler method. Both RNA sequencing and TaqMan®qRT-PCR results in FIGS. 15 and 16 therefore indicate that there was greater inner ear hair cell differentiation in the organoids that were developed using CYC, GANT58 and GANT61 in comparison with organoids developed using the Koehler method. The results therefore indicate that SHH signaling inhibition can increase the number of inner ear hair and neuronal cells derived from preplacodal ectoderm.

Gene ontology functional enrichment analysis of genes was conducted using the program GiTools with false discovery rate correction. The results are presented in FIG. 16.

### Example 18: Development of Sensory Hair Cells of Inner Ear Organoids at Day 33-60

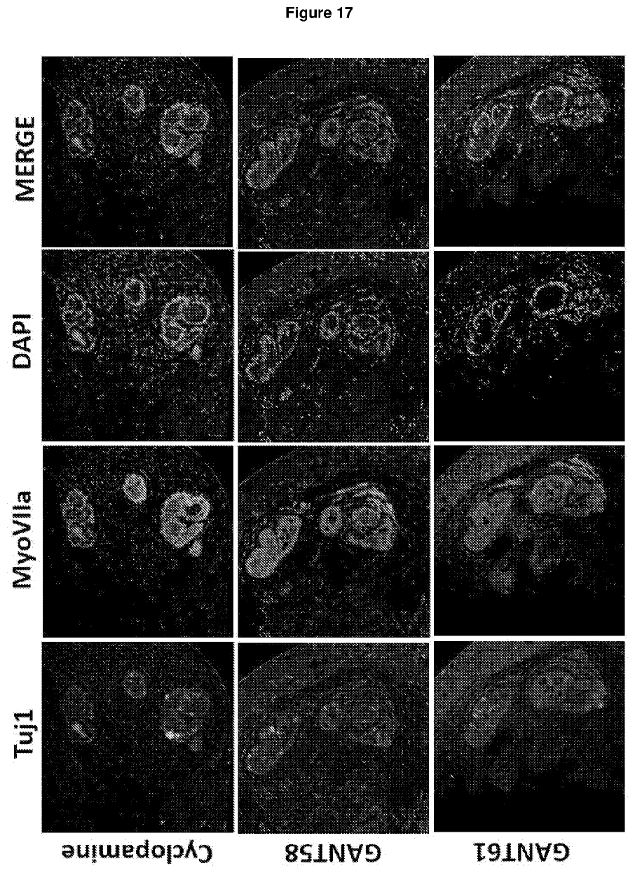

To identify the development of sensory hair cells of inner ear organoids at Day 33-60, the MyoVIIa and Tuj1 expression in organoids was observed by using confocal microscopy. The results are presented in FIG. 17, which compares organoids developed using (a) the method of Example 1 with cyclopamine (Example 2), and (b) the method of Example 1 with GANT58 (Example 3) and GANT61 (Example 4). FIG. 17 shows that sensory hair cells and neuronal cells were present in organoids developed using both SHH inhibitors. An opaque outer cell mass is indicative of complete re-organization, which was found in organoids developed using each of the SHH inhibitors. Sensory hair cell epithelium through the aggregate surface was found in organoids developed using each SHH inhibitor. These results suggest that SHH signalling inhibition can increase the number of inner ear hair cells derived from preplacodal ectoderm.

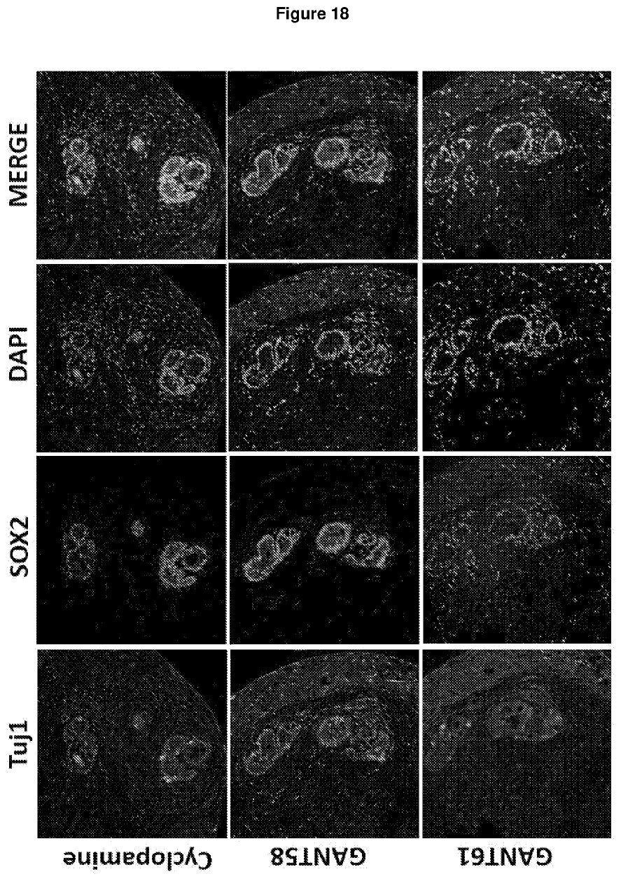

To identify the development of sensory hair cells of inner ear organoids at Day 33-60, the SOX2 and Tuj1 expression in organoids was observed by using confocal microscopy. The results are presented in FIG. 18, which compares organoids developed using (a) the method of Example 1 with cyclopamine (Example 2), and (b) the method of Example 1 with GANT58 (Example 3) and GANT61 (Example 4). FIG. 18 shows that supporting cells and neuronal cells were present in organoids developed using both SHH inhibitors.

1. A method for producing inner ear hair cells, comprising the steps of:
A. culturing otic prosensory vesicles in the presence of a SHH inhibitor in an amount sufficient to partially inhibit the SHH pathway and a culture medium comprising 5-10% of the gelatinous protein mixture secreted by EHS mouse sarcoma cells;
B. removing the SHH inhibitor from the culture in step A; and
C. culturing the cells from step B in a culture medium comprising 5-10% of the gelatinous protein mixture secreted by EHS mouse sarcoma cells to form inner ear hair cells.

2. The method of claim 1 wherein the otic prosensory vesicles are produced by:
AA1. culturing induced pluripotent stem cells under conditions that result in the formation of embryoid bodies from the cultured pluripotent stem cells;
AA2. culturing the embryoid bodies from step AA1 in the presence of an FGF at a concentration of 2-4 ng/mL, and a TGF-β inhibitor at a concentration of 5-10 μM to form non-neural ectoderm cells;
AA3. culturing the non-neural ectoderm cells from step AA2 in the presence of FGF at a concentration of 50-100 ng/mL and a BMP inhibitor at a concentration of 100-200 nM to form pre-otic placodal epithelial cells; and
AA4. culturing the pre-otic placodal epithelial cells from step AA3 in the presence of a WNT agonist at a concentration of 2-3 μM and a cell culture medium comprising 5-10% of the gelatinous protein mixture secreted by Engelbreth-Holm-Swarm (EHS) mouse sarcoma cells to form otic prosensory vesicles.

3. The method of claim 1 wherein the SHH inhibitor is selected from cyclopamine, GANT58 or GANT61.

4. The method of claim 1 wherein the SHH inhibitor is cyclopamine, wherein the cyclopamine is present in step A at a final concentration of 1-2 μM.

5. The method of claim 1 wherein the SHH inhibitor is GANT 61, wherein the GANT61 is present in step A at a final concentration of 1-2 μM.

6. The method of claim 1 wherein the SHH inhibitor is GANT 58, wherein the GANT58 is present in step A at a final concentration of 1-2 μM.

7. The method of claim 1 wherein the inner ear hair cells are inner hair cells.

8. The method of claim 2 wherein the FGF is FGF2.

9. The method of claim 2 wherein the TGF-β inhibitor is SB-431542.

10. The method of claim 2 wherein the BMP inhibitor is LDN-193189.

11. The method of claim 2 wherein the WNT agonist is CHIR-99021.

12. The method of claim 2 wherein step AA1 comprises culturing the pluripotent stem cell in a suitable medium together with a ROCK inhibitor.

13. The method of claim 1 wherein:
a. step A occurs for about 10 days for otic placode and otic vesicles formation;
b. step B occurs for about 15 days for sensory epithelium formation; and
c. step C occurs for about 68 days for hair cell and neural innervation formation until maturation.

14. The method of claim 2 wherein:
a. step AA2 occurs from embryoid body formation from day 0 to day 3, where day 0 is the day on which step AA2 commences for non-neural ectodermal formation;
b. step AA3 occurs from day 4 to day 7 for early pre-otic placodal epithelium formation;
c. step AA4 occurs from day 8 to day 17 for otic placode and otic vesicles formation;
d. step B occurs from day 18 to day 32 for sensory epithelium formation; and
e. step C occurs from day 33 to day 100 for hair cell and neural innervation formation until maturation.

15. The method of claim 13 wherein maturation occurs between day 60-200.

16. A composition comprising inner ear hair cells produced by the methods in claim 1.

17. A method of treating sensorineural hearing loss in a subject in need thereof comprising administering an effective amount of the composition of claim 16 to the subject.

18. A method for assessing the ototoxicity or therapeutic effectiveness of a test agent comprising a step of treating with a test agent a population of inner ear hair cells or an organoid comprising inner ear hair cells of produced by the methods of claim 1.

19. The use of the composition of claim 16 in the manufacture of a medicament for the treatment of sensorineural hearing loss in a subject in need thereof.

20. A method of regenerating inner ear hair cells in a subject comprising the step of administering a SHH inhibitor to the subject's inner ear.
